(19) United States
(12) Patent Application Publication
(10) Pub. No.: US 2008/0044439 A1
(43) Pub. Date: Feb. 21, 2008

(54) Compositions and Methods for Preventing and Treating Skin and Hair Conditions

(76) Inventor: Nathaniel E. David, Los Angeles, CA (US)

Correspondence Address: WILSON SONSINI GOODRICH & ROSATI, 650 PAGE MILL ROAD, PALO ALTO, CA, 94304-1050, US

(21) Appl. No.: 10/591,934

(22) PCT Filed: Feb. 25, 2005
(86) PCT No.: PCT/US05/06300

§ 371 Date: Apr. 23, 2007

(30) Foreign Application Priority Data
Mar. 11, 2004 (US) 10/799540
Mar. 12, 2004 (US) 10/799867
Mar. 26, 2004 (US) 10/810391

(51) Int. Cl. A61K 39/00; A61K 31/415; A61K 31/4164; A61K 31/435; A61K 39/38; A61P 17/00; A61K 39/08; A61K 31/517; A61K 36/00

(52) U.S. Cl. 424/239.1; 424/184.1; 424/725; 514/266.1; 514/277; 514/396; 514/406

(57) ABSTRACT

The present invention discloses compositions and methods for the prevention and treatment of skin and hair diseases, such as, for example, alopecia, psoriasis, and keloids. The present invention also relates to compositions and methods for altering skin coloration. The present invention also relates to methods for enhancing the effect of a neurotoxin.

### BACKGROUND OF THE INVENTION

The present invention relates to compositions and methods for preventing and treating skin and hair conditions.

The skin is the second largest organ in the body and is of primary importance to the survival of a mammal. The skin rests on subcutaneous tissue largely composed of a loose mesh of collagen fiber, fat cells, and muscle tissue. An average adult has over 3,000 square inches of skin surface area. Overall, fat-free skin accounts for at least 6 percent of an individual's total weight. The density of structures in the skin varies considerably depending on its location. But on average, one square centimeter of skin contains about 10 hair follicles, 15 sebaceous glands, 100 sweat glands, half a meter of blood vessels, 2 meters of nerves with 3,000 sensory cells at the ends of nerve fibers, 200 nerve endings to record pain, 25 pressure receptors for the perception of tactile stimuli, 2 sensory receptors for cold, and 12 sensory receptors for heat.

The skin of a mammal is derived from ectoderm and mesoderm layers of an embryo. These two layers give rise to the epidermis and dermis, respectively. The ectoderm and mesoderm layers also give rise to specialized appendages including sensory nerves, sweat glands, and hair follicles. Thus, the skin and hair follicles are physiologically related.

The skin serves various functions including, but not limited to, providing flexible physical support, maintaining constant temperature, excreting waste materials such as salts and water, producing vitamins by photochemical reactions in the skin, sensory functions, providing protection against the excesses of ultraviolet light by pigmentation such as melanin, providing protection of organs, preventing absorption of unwanted or dangerous chemicals, and providing an immunological defense.

Hair serves similar functions. The main function of hair is to provide protection against heat loss. Hair may also act to protect the epidermis from minor abrasions and from ultraviolet light. In addition, hair may provide indication of sexual development. It may also play an important role in attracting mates by indicating the general health and vitality of an individual. Furthermore, certain body parts may contain specialized hairs. Specialized hair such as eyebrows and eyelashes act to protect the eyes by channeling or sweeping away fluids, dust and debris. Nasal hair act to trap air borne foreign particles before they reach the lungs. These specialized hairs and other hair follicles have a highly developed nerve network around them that can provide sensory, tactile information about the environment.

There are many conditions that affect skin and hair. Such conditions include, but are not limited to, acne, scarring, vitiligo, and hair loss. It would be desirable to identify novel methods and compositions for preventing and/or treating skin and hair conditions.

Skin color is a conspicuous way in which humans vary. Today, many people use tattoos to alter skin coloration for aesthetic and cosmetic reasons. For example, some individuals tattoo permanent makeup. Others use tattooing to simulate natural pigmentation. Tattooing can also be used as part of an initiation ceremony to a social group.

Whatever the reason is, tattooing has become a common procedure. It is approximated that over 10 million Americans have at least one tattoo, and that close to 4,000 tattoo studios currently operate in the United States. Yet, estimates suggest that almost 50 percent of all those who get tattoos later decide to remove them.

Tattoo removal can be painful, expensive and often results in scarring or discoloration of the skin. The most commonly used color alteration procedures these days are excision, dermabrasion, laser therapy, cryosurgery, grafting, camouflaging, scarification, and salabrasion. However, no matter which procedure is used, the average tattoo requires 8-12 treatments before it is substantially removed. Thus, it is desirable to identify novel methods and compositions to reduce the number of treatments for tattoo removal, alleviate the pain associated with tattoo removal, and enhance the results.

Neurotoxins are also used for the treatment and prevention of various diseases as well as for cosmetic applications. A commonly used neurotoxin is botulinum toxin type A. Botulinum toxin type A is a member of a family of toxins that was first discovered by Professor Emile Pierre van Ermengem in lb 1895. The botulinum toxins were isolated and purified in the 1920by Dr. Herman Sommer at the University of California, San Francisco. Botulinum toxin type A was separated out from the other types of botulinum toxins in the 1960's. By the 1970's, type A was found to be effective in treating neuronal disorders, such as those related to involuntary crossing of the eyes and related to neck and head spasms. Since then, other botulinum toxin types (e.g., botulinum toxin types B, C, D, E, F, and G) have also been isolated and have shown to be effective in the treatment of various conditions. Today, botulinum toxin type A is the most commonly used botulinum toxin and is approved for the treatment of brow wrinkle removal, and optical conditions, such as blepharospasm, strabismus, and Duane's syndrome. However, the use of neurotoxins, including botulinum toxin type A, may be risky and may cause severe side effects. Examples of side effects caused by botulinum toxin type A include, but are not limited to, flu like symptoms, weakness in the group of muscles being treated, difficulty swallowing, collapsed lung, etc.

Thus, it would be desirable to identify compositions and methods that increase the effect of a neurotoxin treatment (e.g., increase the duration of effect of a neurotoxin treatment), thereby reducing the amount of neurotoxin administered per application or the number of applications per treatment cycle. An additional benefit of such compositions and methods includes reducing the antigenicity to the neurotoxin.

### SUMMARY OF THE INVENTION

The present invention relates to methods for treating and/or preventing skin and hair conditions.

In particular, the present invention relates to methods for treating and/or preventing hair loss in a patient by administering to such patient an effective amount of one or more p38 inhibitors. The p38 inhibitors are preferably administered locally to a region requiring hair regeneration or prevention of hair loss. More preferably, the p38 inhibitors are administered topically, transdermally or subcutaneously.

The present invention also relates to methods for treating and/or preventing skin conditions, such as, for example, vitiligo and acne, and acne scars by administering to such a patient an effective amount of one or more p38 inhibitors. Again, the p38 inhibitors are preferably administered locally. More preferably, the p38 inhibitors are administered topically, transdermally or subcutaneously.

Examples of p38 inhibitors include, but are not limited to, pyridinylimidazoles, substituted pyrazoles, substituted pyridyls, quinazoline derivatives, aryl ureas, heteroaryl analogues, substituted imidazole compounds, and substituted triazole compounds.

The present invention also involves methods and compositions for altering skin coloration, and, in particular, tattoo removal. In preferred embodiments, the methods herein provide administering to a dermal region an effective amount of a cytokine (e.g., a tumor necrosis factor, interferon, or interleukin). The cytokine administered is preferably not a GM-CSF. The cytokine administered is preferably a tumor necrosis factor, an interferon, or an interleukin. More preferably, the cytokine administered is TNF-α, IFN-α, and/or IL-1.

One or more cytokines is preferably administered locally. Local administration is preferably made by topical, subcutaneous, or transdermal administration. The cytokines can be administered as a single dose, multiple doses, in combination with other agents, and/or in combination with other treatments.

In some embodiments, the dermal region being treated with a cytokine is also treated with a color alteration treatment. Examples of color alteration treatments include, but are not limited to, excision, dermabrasion, laser therapy, cryosurgery, grafting, camouflaging, scarification, and salabrasion. In preferred embodiments, the color alteration treatment is a laser therapy. In some embodiments, the cytokine is administered prior to the color alteration treatment. In some embodiments, the cytokine is administered after the color alteration treatment. In some embodiments, the cytokine is administered during a color alteration treatment.

The present invention also relates to compositions and methods that increase the efficacy of a neurotoxin treatment. Enhancing the efficacy of a neurotoxin treatment can take place, or example, by inhibiting or delaying neurojunction repair or by delaying, reducing, inhibiting or interfering with the process neuronal growth and/or axonal sprouting.

A neurojunction can be any junction with a neuron. In preferred embodiments, the neurojunction is a neuromuscular junction between a neuron and a muscle cell. In such junctions, neurotransmission is usually conducted by a neurotransmitter (e.g., Acetylcholine (ACh)). Repair and/or reconstruction of a neurojunction typically involve neuronal cell growth and/or axonal sprouting.

In some embodiments, the methods herein include administering locally to a target region of a mammal a neurotoxin and a neuron growth inhibitor. The neurotoxin is preferably a botulinum toxin selected from the group consisting of botulinum toxin types A, B, C, D, E, F, and G. More preferably, the neurotoxin is botulinum toxin is of type A. The neuron growth inhibitor may be any agent that inhibits neuronal cell growth and/or axonal sprouting. In preferred embodiments, the neuronal growth inhibitor is selected from the group consisting of a Trk receptor inhibitor, a Ras inhibitor, a Raf inhibitor, a Rap-1 inhibitor, a MEK inhibitor, an ERK inhibitor, a PKA inhibitor, a PKC inhibitor, a p53 inhibitor, a growth factor inhibitor, or an inhibitor of any activator or effector of any of the above. In preferred embodiments, the neuron growth inhibitor is a MEK inhibitor or a Raf inhibitor (e.g., a b-Raf inhibitor). A MEK inhibitor is preferably selected from the group consisting of PD98059, U0126, PD 184352, 2-Cholor-3-(N-succinimidyl)-1,4-naphthoquinone, PD 184352, ARRY-142886, tricyclic flavone, and 2-(2-amino-3-methoxyphenyl)-4-oxo-4H-[1]benzopyran.

A Raf inhibitor is preferably Rheb, BAY-43-9006 or a Raf kinase protein inhibitor (RKPI).

A neurotoxin can be administered prior to, simultaneous with, or after administration of a neuron growth inhibitor. In preferred embodiments, the neurotoxin is administered after the administration of the neuron growth inhibitor.

In preferred embodiment, both the neurotoxins and the neuron growth inhibitors are administered locally. Means for localized administration include any method known in the art, but preferably by topical, transdermal, subdermal, subcutaneous, or intramuscular administration.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates p38 inhibitor BIRB-796.

FIG. 2 illustrates p38 inhibitor CNI-1493.

FIG. 3 illustrates p38 inhibitor RDP-58.

FIG. 4 illustrates p38 inhibitor VX-745.

FIG. 5 illustrates signaling pathways of the immune system.

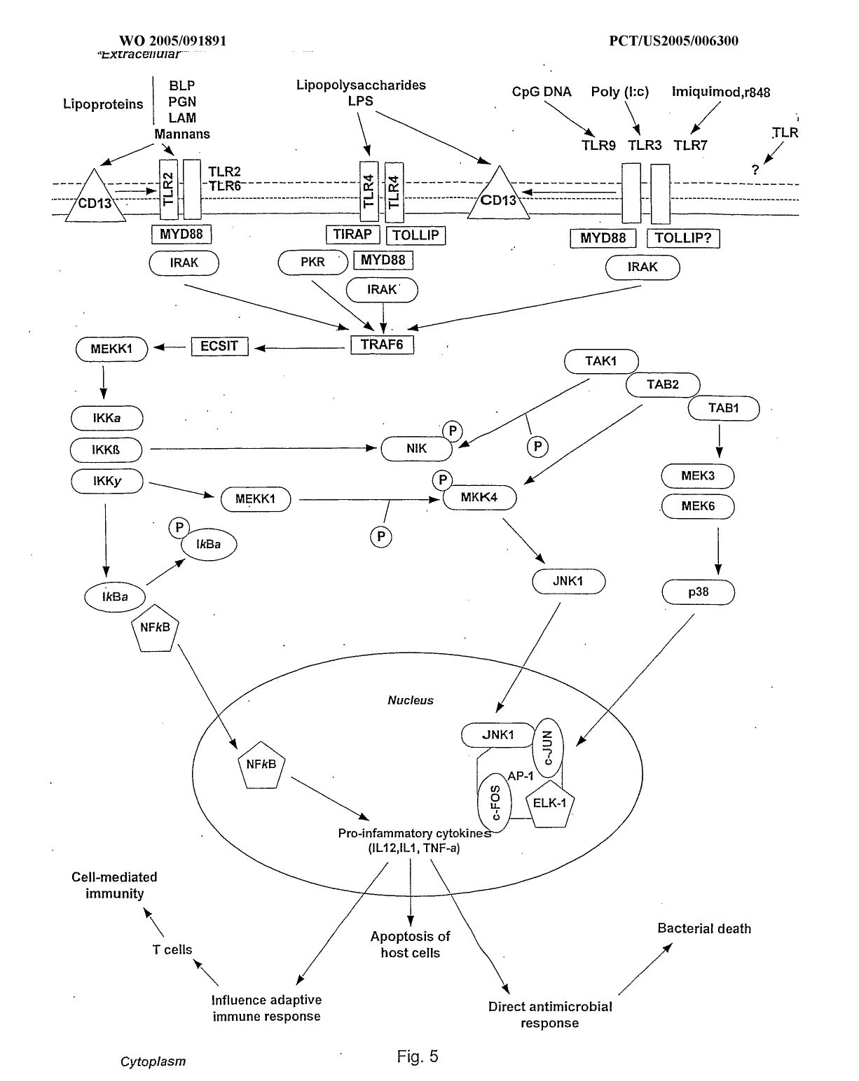

FIG. 6 illustrates a typical signal transduction pathway via MAPK.

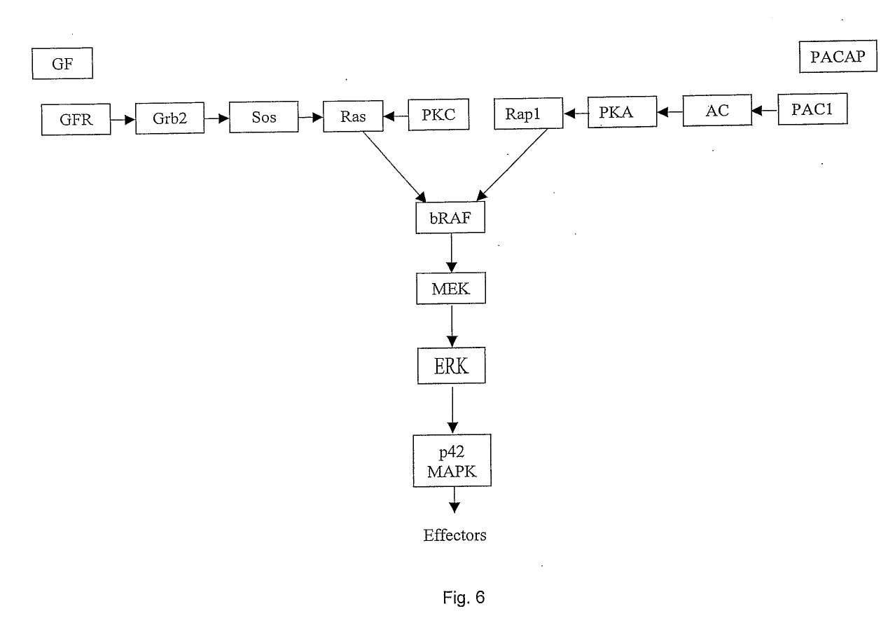

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention relates to compositions and methods for preventing and treating skin and hair conditions. The compositions of the present invention include at least one p38 MAP kinase (referred to herein as “p38 ”) inhibitor. The term “p38 ” as used herein, refers to all isoforms, splicing variants, homologues, fragments, metabolites, prodrugs, and mimetics of p38 , both naturally occurring and synthetic. The term “p38 inhibitor,” as used herein, refers to any agent that blocks, diminishes, inhibits, hinders, limits, decreases, reduces, restricts or interferes with the activity of endogenous p38 . A p38 inhibitor can also function upstream or downstream of p38 to downregulate the amount or function of p38.

p38 is a stress-activated protein. p38 can be activated by, for example, UV light, heat, chemical or osmotic shock, IL-1, TNF, and endotoxins. p38 is one of three families of MAP kinases: the extracellular regulated kinases (ERKs), the c-Jun NH2 terminal kinases or stress activated protein kinases (JNKs or SAP kinases), and the p38 MAP kinases. A distinguishing feature of each of these kinase families is that the ERKs have a TEY amino acid motif, the JNKs or SAP kinases have a TPY amino acid motif, and the p38 MAP kinases have a TGY amino acid motif.

The p38 family includes four different isoforms: p38 α MAP kinase (p38 α), p38 β MAP kinase (p38 β), p38 γ MAP kinase (p38 γ), and p38 δ MAP kinase (p38 δ). p38 α is expressed ubiquitously. A shorter C-terminal truncated form of p38 α known as Mxi-2 has also been identified in a yeast two-hybrid screen based on its association with the transcription factor Max. p38 β has been shown to have an additional isoform, p38 β2 that lacks the 8 amino acid insertion found in p38 β. Between these two variants p38 β2 is believed to be the major form as p38 β is catalytically less active. p38 γ and p38 δp0 are 63% and 61% identical to p38 α, respectively. p38 γ is expressed predominantly in skeletal muscle wherein p38 δ is expressed predominantly in testes pancreas, prostate, small intestine, and endocrine tissue.

All p38 homologues and splice variants contain a 12 amino acid activation loop between kinase domain VII and kinase domain VIII. The activation loop includes a Thr-Gly-Tyr motif. Dual phosphorylation of both Thr-180 and Tyr-182 (p38 α numbering) in the TGY motif is essential for the activation of p38 resulting in >1000 fold increase in specific activity of these enzymes. Dual phosphorylation can be effected by MKK6, MKK3 and other members of the MAPKK (mitogen activating protein kinase kinase) family and MAPKKK (mitogen activating protein kinase kinase kinase) family, also referred to as the MAP3K family. In particular, MEKK4/MTK1, ASK1, and TAK1 have been identified as upstream activators of MAP3K. Also, TNF-stimulated activation of p38 α is believed to be mediated via recruitment of TRAF2 (TNF receptor associated factor) and the Fas adaptor protein, Dazz, which results in the activation of ASK1 and subsequently p38 and JNK. Also, TAK has been shown to activate MKK6 in response to TGF-β and is believed to be associated with TRAF6 in an IL-1-dependent manner suggesting involvement of TAK1 in IL-1-mediated p38 activation. Additionally, mixed lineage of kinase-3 physically associated with MKK3 and MKK6 is believed to be involved in activation of p38 by Ste-20-linked kinases. Also, MEKK3, small G proteins of the Rho family, and active forms of Cdc42 and Rac1 in mammalian cells have also been shown to activate the p38 pathways (the latter via p21-activation kinase).

Thus, p38 is a key control point in the cellular immune system. In particular, p38 exerts its effects by regulating the production of cytokines. p38 is activated by phosphorylation on Thr-180 and Tyr-182 by MEKs (MKK3 or MKK6), and in response to that, p38 phosphorylates MAPJAP2 kinase, which relieves post-transcriptional repression of TNF-α and IL1 transcripts by phosphorylating (and thus inactivating) a AU-rich binding protein that binds to the 3′-UTR of the TNF and IL1 mRNAs. Because multiple stress pathways are able to activate p38 , p38 inhibition is able to broadly suppress cytokine (e.g., TNF-α, IFN-α, IL1) production and its resulting activation of the immune system.

To date, there have been several mechanisms and numerous compounds suggested for the inhibition of p38 . Compounds that have been suggested for the inhibition of p38 include pyridinylimidazoles. See Young P. R., et al., (1997) J. Biol. Chem. 272, 12116-12121; see also Bender, P. E., (1985) J. Med. Chem. 28, 1169-1177. Examples of pyridinylimidazoles that may inhibit p38 include 6-(4′-fluorophenyl)-5-(4′-pyridyl)-2,3-dihydroimidazo(2,1-b)-thiazole and its metabolites (sulfoxide, sulfone), analogues, fragments, and mimetics. It has further been suggested that the minimal structure of pyridinylimidazoles, 4-(pyridin-4-yl)-5-phenylimidazole, may be sufficient to inhibit p38 . See Gallagher, T F, et al., (1997) Bio-org. Med. Chem. 5, 49-64.

Certain 1,5-diaryl-substituted pyrazole compounds have also been suggested as p38 inhibitors. Such substituted pyrazole compounds are disclosed in U.S. Pat. No. 6,509,361, assigned to Pharmacia Corporation, incorporated herein by reference for all intended purposes. Additional pyrazole derivatives that inhibit p38 are disclosed in U.S. Pat. No. 6,335,336, assigned to G. D. Searle & Co., incorporated herein by reference for all intended purposes.

Other p38 inhibitors include substituted pyridyl, such as those disclosed in U.S. Patent Application Publication No. 2003/0139462, incorporated herein by reference for all intended purposes.

Additional p38 inhibitors are those disclosed in U.S. Pat. No. 6,610,688, assigned to Sugen, Inc., incorporated herein by reference for all intended purposes.

Quinazoline derivatives may also function as p38 inhibitor. Examples of quinazoline derivatives that are p38 inhibitors are disclosed in U.S. Pat. Nos. 6,541,477 and 6,184,226, assigned to Scios Inc., incorporated herein by reference for all intended purposes, and U.S. Pat. Nos. 6,509,363 and 6,635,644, assigned to Vertex Pharmaceuticals Inc., incorporated herein by reference for all intended purposes.

Aryl ureas and heteroaryl analogues may also function as p38 inhibitors. Examples of aryl ureas and heteroaryl analogues that are p38 inhibitors are disclosed in U.S. Pat. No. 6,344,476, assigned to Bayer Corp., incorporated herein by reference for all intended purposes. WO99/32110, published Jul. 1, 1999, describes heterocyclic ureas as p38 kinase inhibitors. WO99/32463, published Jul. 1, 1999, describes urea compounds that inhibit p38 kinase. WO98/52558, published Nov. 26, 1998, describes urea compounds for the inhibition of p38 kinase. WO99/00357, published Jan. 7, 1999, describes the use of urea compounds as inhibitors of p38 kinase. WO99/58502, published Nov. 18, 1999, describes urea compounds as inhibitors of p38 kinase. These and all other references mentioned herein are incorporated by references for all purposes.

Substituted imidazole compounds and substituted triazole compounds may also function as p38 inhibitors. Such compounds are disclosed in U.S. Pat. Nos. 6,560,871 and 6,599,910, respectively, which incorporated herein by reference for all intended purposes.

Additional p38 inhibitors include RWJ-67657 (RW Johnson Pharmaceutical Research Institute); RDP-58 (SangStat Medical Corp.); RDP-58; Scios-323 (Scios Inc.); Scios-469 (Scios Inc.); MKK3/MKK6 inhibitors (Signal Research Division); p38 /MEK modulators (Signal Research Division); SB-210313 analogs, SB-220025, SB-238039, HEP-689, SB-203580, SB-239063, SB-239065, SB-242235 (SmithKline Beecham Pharmaceuticals); VX-702 and VX-745 (Vertex Pharmaceuticals Inc.); AMG-548 (Amgen Inc.); Astex p38 kinase inhibitors (Astex Technology Ltd.); RPR-200765 analogs (Aventis SA); Bayer p38 kinase inhibitors ( Bayer Corp.); BIRB-796 (Boehringer Ingelheim Pharmaceuticals Inc.); Celltech p38 MAP kinase inhibitor (Celltech Group plc.); FR-167653 (Fujisawa Pharmaceutical Co. Ltd.); 681323 and SB-281832 (GlaxoSmithKline plc); LEO Pharmaceuticals MAP kinase inhibitors (LEO Pharma A/S); Merck & Co. p38 MAP kinase inhibitors (Merck research Laboratories); SC-040 and SC-XX906 (Monsanto Co.); Novartis adenosine A3 antagonists (Novartis AG); p38 MAP kinase inhibitors (Novartis Pharma AG); CP-64131 (Pfizer Inc.); CNI-1493 (Picower Institute for Medical Research); RPR-200765A (Rhone-Poulenc Rorer Ltd.); and Roche p38 MAP kinase inhibitors and Ro-320-1195 (Roche Bioscience).

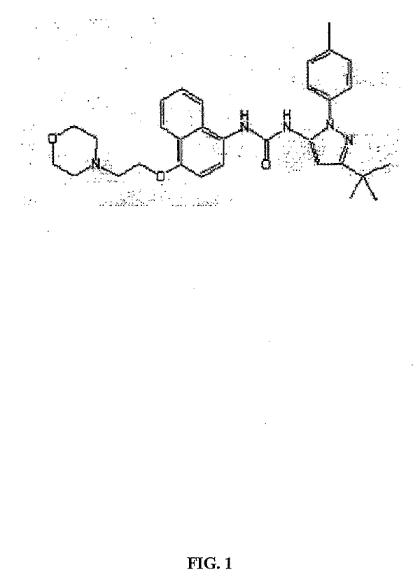

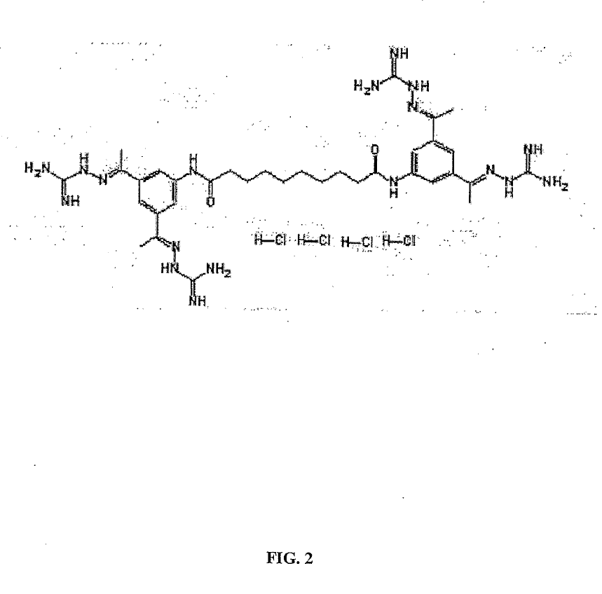

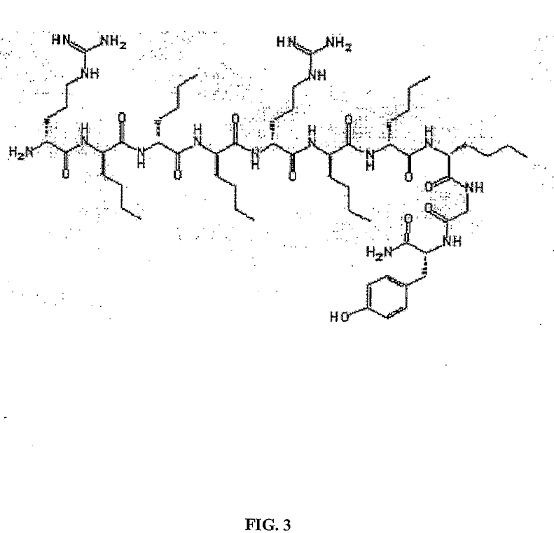

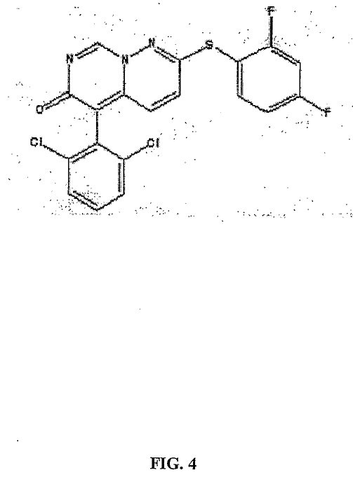

In preferred embodiments, the p38 inhibitor is RDP-58 (SangStat Medical Corp.), AMG-548 (Amgen Inc.), BIRB-796 (Boehringer Ingelheim Pharma.), CNI-1493 (Picower Institute for Medical Research), VX-702 or VX-745 (Vertex Pharmaceuticals Inc.). FIG. 1 illustrates p38 inhibitor BIRB-796. FIG. 2 illustrates p38 inhibitor CNI-1493. FIG. 3 illustrates p38 inhibitor RDP-58. FIG. 4 illustrates p38 inhibitor VX-745.

The present invention also relates to compositions that include at least one p38 inhibitor, and that may optionally include one or more additional active agents. Active agents can include, for example, anti-inflammatory agents, immunomodulators, antibacterial agents, antiviral agents, and/or antifungal agents.

Anti-inflammatory agents include, but are not limited to, pyrazolones, fenamate, diflunisal, acetic acid derivatives, propionic acid derivatives, oxicams, mefenamic acid, Ponstel™, meclofenamate, Meclomen™, phenylbutazone, Butazolidin™, diflunisal, Dolobid™, diclofenac, Voltaren™, indomethacin, Indocin™, sulindac, Clinoril™, etodolac, Lodine™, ketorolac, Toradol™, nabumetone, Relafen™, tolmetin, Tolectin™, ibuprofen, Motrin™, fenoprofen, Nalfon™, flurbiprofen, Ansaid™, carprofen, Rimadyl™, ketoprofen, Orudis™, naproxen, Anaprox™, Naprosyn™, piroxicam, and Feldene™.

The term “immunomodulator” as used herein includes cytokines, stem cell growth factors, lymphotoxins, co-stimulatory molecules, hematopoietic factors, and synthetic analogs of these molecules. Examples of immunomodulators include tumor necrosis factor, interleukins (e.g., interleukin-1 (IL-1), IL-2, IL-3, IL-4, IL-5, IL-6, IL-7, IL-8, IL-9, IL-10, IL-11,IL-12IL-13, IL-14, and IL-15), colony stimulating factors (e.g., granulocyte-colony stimulating factor and granulocyte macrophage-colony stimulating factor), interferons (e.g., interferons-α, β, γ, δ, ε, Ω, T), the stem cell growth factor designated “S1 factor,” erythropoietin, and thrombopoietin. Additional examples of immunomodulators include, but are not limited to, azathioprine (Imuran), 6-mercaptopurine (6-MP, Purinethol), cyclosporine (Sandimmune), and methotrexate.

Examples of antibacterial agents include, but are not limited to, a tetracycline, a sulfa drug, a penicillin, a quinolone, a cephalosporin, and mixtures thereof. Exemplary tetracyclines include doxycycline and minocycline. An exemplary sulfa drug includes sulfacetamde. An exemplary cephalosporin includes cephalexin (commercially available as KEFLEX). Exemplary quinolones include the floxacins, such as loemfloxacin, ofloxacin, and ciprofloxacin.

Examples of antiviral agents include, but are not limited to, acyclovir, tamvir, penciclovir, and the like, and mixtures thereof.

Examples of anti-fungal agents include but are not limited to, farnesol, econazole, fluconazole, clotrimazole, ketoconazole, calcium or zinc undecylenate, undecylenic acid, butenafine hydrochloride, ciclopirox olainine, miconazole nitrate, nystatin, sulconazole, terbinafine hydrochloride, and the like, and mixtures thereof.

It should be readily understood that any salts, isomers, prodrugs, metabolites, or other derivatives of these anti-microbial agents may also be included as the anti-microbial agent in accordance with the invention.

A pharmaceutical composition of the present invention may be formulated to be suitable for application in a variety of manners, for example, in a cream for topical application to the skin (e.g., for alopecia), in a wash, in a douche, in a powder for chaffing (e.g., for dermatitis), in a liquid, in a dry formulation (e.g., as a bath salt or bath powder), and the like. Other formulations will be readily apparent to one skilled in the art. In preferred embodiments, the compositions herein are preferably formulated for local administration. Preferably, the compositions are formulated for topical, subcutaneous or transdermal administration.

When formulated as an ointment, the active ingredient (e.g., a p38 inhibitor) can be employed, for example, with either paraffinic or a water miscible ointment base. Alternatively, the active ingredients can be formulated in a cream with an oil-in-water cream base. If desired, the aqueous phase of the cream base can include, for example at least 30% w/w of a polyhydric alcohol such as propylene glycol, butane-1,3-diol, mannitol, sorbitol, glycerol, polyethylene glycol and mixtures thereof.

The topical formulations can desirably include a compound that enhances absorption or penetration of the active ingredient through the skin or other affected areas. Examples of such dermal penetration enhancers include dimethylsulfoxide and related analogs.

The pharmaceutical compositions herein may also include, for example, antioxidants (e.g., vitamin E); buffering agents; lubricants (e.g., synthetic or natural beeswax); sunscreens (e.g., para-aminobenzoic acid); and other cosmetic agents (e.g., coloring agents, fragrances, oils, essential oils, moisturizers or drying agents). Thickening agents (e.g., polyvinylpyrrolidone, polyethylene glycol or carboxymethyicellulose) may also be added to the compositions.

The carriers utilized in the pharmaceutical compositions of the present invention may be solid-based dry materials for use in powdered formulations or may be liquid or gel-based materials for use in liquid or gel formulations. The specific formulations depend, in part, upon the routes or modes of administration.

Typical carriers for dry formulations (e.g., bath salts) include, but are not limited to, trehalose, malto-dextrin, rice flour, micro-crystalline cellulose (MCC), magnesium sterate, inositol, fructo-oligosaccharides FOS, gluco-oligosaccharides (GOS), dextrose, sucrose, talc, and the like carriers. Where the composition is dry and includes evaporated oils that produce a tendency for the composition to cake (i.e., adherence of the component spores, salts, powders and oils), it is preferable to include dry fillers which both distribute the components and prevent caking. Exemplary anti-caking agents include MCC, talc, diatomaceous earth, amorphous silica and the like, typically added in an concentration of from approximately 1% to 95% by-weight.

Suitable liquid or gel-based carriers are well-known in the art (e.g., water, physiological salt solutions, urea, methanol, ethanol, propanol, butanol, ethylene glycol and propylene glycol, and the like). Preferably, water-based carriers are approximately neutral pH.

Suitable carriers include aqueous and oleaginous carries such as, for example, white petrolatum, isopropyl myristate, lanolin or lanolin alcohols, mineral oil, fragrant or essential oil, nasturtium extract oil, sorbitan mono-oleate, propylene glycol, cetylstearyl alcohol (together or in various combinations), hydroxypropyl cellulose (MW=100,000 to 1,000,000), detergents (e.g., polyoxyl stearate or sodium lauryl sulfate) and mixed with water to form a lotion, gel, cream or semi-solid composition. Other suitable carriers comprise water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether and water. For example, emulsions containing water, glycerol stearate, glycerin, mineral oil, synthetic spermaceti, cetyl alcohol, butylparaben, propylparaben and methylparaben are commercially available. Preservatives may also be included in the carrier including methylparaben, propylparaben, benzyl alcohol and ethylene diamine tetraacetate salts. Well-known flavorings and/or colorants may also be included in the carrier. The composition may also include a plasticizer such as glycerol or polyethylene glycol (MW 400 to 20,000). The composition of the carrier can be varied so long as it does not interfere significantly with the pharmacological activity of the active ingredient (p38 inhibitor).

The compositions and pharmaceutical compositions herein may be used to prevent and treat skin and hair conditions.

Examples of skin conditions include, but are not limited to, acne, acne scars, scleroderma, psoriasis, atopic dermatitis, vitiligo, keloid, hypertrophic scars, and vascularity. Skin conditions also include any condition that causes irritation, inflammation, infection or discoloration of skin.

The term acne refers to plugged pores (blackheads and whiteheads), pimples, papules, pustules, macules, cysts or nodules. Acne can occur on all body parts and can affect people of all ages. While not life threatening, acne can often lead to scarring which may be permanent.

Acne may result from hair follicle blockage. The hair follicle blockage allows for sebum (oil), which normally drains to the surface of the skin, to aggregate and for bacteria to grow. It is postulated that androgen may be involved in causing acne, and that the sebaceous glands of people with acne react differently, or excessively, to normal levels of androgen hormones. Normally, skin cells in the follicles grow, mature, die, flake off and are carried to the surface of the skin by the flow of sebum. However, for acne patients, it is suggested that dead cells fail to be carried to the surface and instead block the inside of the follicle, trapping oil and bacteria (e.g., P. acnes), which in turn lead to acne.

When the trapped sebum and bacteria stay below the skin surface, a whitehead is formed. On the other hand, when the trapped sebum and bacteria open to the surface they turn black due to melanin, the skin's pigment, and a blackhead is formed. Blackheads can last for a long time because the contents very slowly drain to the surface.

Whiteheads and blackheads are also referred to as comedo. A comedo is a sebaceous follicle plugged with sebum, dead cells from inside the sebaceous follicle, tiny hairs, and sometimes bacteria. Neither blackheads nor whiteheads should be squeezed or opened, unless it is done under sterile conditions. This is to prevent subsequent skin infection by bacteria (e.g., staphylococci).

In more severe forms of the disease papules, pastures, nodules, and cysts may be formed. A papule is a small, solid lesion slightly elevated above the surface of the skin. A papule is usually less than 5 mm across. A papule is believed to be caused by localized cellular reaction to the process of acne. A group of papules and microcomedones (blackheads and whiteheads) may be almost invisible but can create a bumpy appearance to the skin.

Like a papule, a nodule is a solid, dome-shaped or irregularly-shaped lesion. However, unlike a papule, a nodule is characterized by inflammation that extends into deeper layers of the skin and may cause tissue destruction and/or scarring. A nodule may be very painful. Nodular acne is a severe form of acne that may not respond to therapies other than isotretinoin.

A pustule is a dome-shaped lesion that contains pus. The pus usually consists of a mixture of white blood cells, dead skin cells, and bacteria. It is common for a pustule that forms over a sebaceous follicle to have a hair in its center. Acne pustules that heal without progressing to cystic form usually do not leave scars.

A macule is the temporary red spot left by a healed acne lesion. A macule is generally flat, red or red-pink, and has a well defined border. A macule may persist for days or weeks before disappearing. When a number of macules are present at one time they can contribute to the “inflamed face” appearance of acne.

A cyst is a sac-like lesion containing liquid or semi-liquid material. The liquid often consists of white blood cells, dead cells, and bacteria. A cyst is larger than a pustule and may be severely inflamed down into deeper layers of the skin. Like nodules, a cyst may be very painful and may result in scarring.

Cysts and nodules often occur together in a severe form of acne called nodulocystic. Systemic therapy with isotretinoin is sometimes the only effective treatment for nodulocystic acne.

Thus, the present invention involves administering to a patient suffering from or susceptible to acne an effective amount of one or more of the compositions herein. Such compositions include at least one p38 inhibitor. Such compositions are preferably administered locally (e.g., topically, transdermally, or subcutaneously). Any composition herein can be administered independently or in combination with one or more additional agents or treatments. Such agents and/or treatments include, but are not limited to, retinoids, antibiotics, oral contraceptives, Accutane, laser treatment (e.g., Smoothbeam), isotretinoin, etc. The p38 inhibitor may be administered prior to, simultaneous with, or after the administration of additional agents and/or treatments. Preferably, the p38 inhibitor will be administered prior to the administration of the additional agent (e.g., a retinoid, an antibiotic or isotretinoin).

The present invention also contemplates the prevention and/treatment of acne scars. Scars, also known medically as cicatrix, are marks left by a healed wound, burn, or incision, and are composed of tough fibrous tissue. There are many forms of scars, including but not limited to, acne scars, keloids, hypertropic scars, pigmentary scars, hormone induced scars, animal bite scars, etc.

Acne scars are a unique form of scars that can occur anywhere on the body. Acne scars can be of various shapes, sizes, and depth. It is thought that acne scars are caused by the activation of the immune system in fighting acne bacteria. Generally, humans and other mammals recognize invading microorganisms by recognizing their microbial patterns, e.g., (1) LPS—lipopolysaccharide, mannose, fucose, and other sugar residues, (2) techoid acid, or (3) N-formyl peptides. These, and other, microbial patterns are recognized by pattern recognition molecules (PRMs) or pattern recognition receptors (PRRs). Examples of PRR's include, f-Met-Leu-Phe receptors, which bind to N-formyl peptides and attract neutrophils; complement receptors (CRs), which bind to complement components such as C3b and C4b; macrophage mannose receptors, which bind to mannose residues commonly present on surface of microorganisms; scavenger receptors, which recognize certain anionic polymers and acetylated low-density lipoproteins; and CD14 receptors on the surface of phagocytes, which allow for the recognition of LPS.

It is postulated that acne bacteria activates the innate immune system via activation of the f-Met-Leu-Phe receptors. Activation of the innate immune system by f-Met-Leu-Phe receptors activates p38 , which has also been shown to be present in scar formation. Thus, by inhibiting p38 , it may be possible to reduce scarring that results from acne or other effects resulting from the activation of the innate immune system. Activation of p38 can be temporary or long term (even permanent).

Current treatments for acne scars include, but are not limited to, dermabrasion, laser resurfacing, chemical peels, punch techniques, subcision, and augmentation. Dermabrasion involves removal of damaged skin using a quickly rotating diamond edged wheel or other abrasive device. Depending on how coarse the wheel or the device is, one can control the amount of skin that is removed. Laser resurfacing involves the use of a laser to remove skin so new skin can form in its place. Common lasers used include the CO₂ laser and the erbium (YAG) laser. Chemical peels involve the application of different types of acid to the skin in order to remove the top layer so that a smoother layer can surface. Punch techniques include: punch replacement, punch excision, and punch elevation. Punch replacement involves the removal of pitted scar with a hair-transplant type punch, which is then replaced with a skin graft, usually from behind the ear. This is usually the most successful method for removal of deep scars. Punch excision involves the removal of a pitted scar. The wound is then closed and allowed to heal. Finally, punch elevation involves cutting the scar loose from the bottom, but not discarding it. The scar is thus allowed to float up to the level of surrounding skin. Subcision involves detaching a scar from deeper tissue, which allows a pool of blood to form under the scar. The blood clot then helps form connective tissue under the scar, leveling it with the surface. Furthermore, augmentation involves injecting material, such as collagen and/or fat, under the scar to bring it to surface level (may follow subcision).

Thus, the present invention relates to the prevention and/or treatment of scars, or more preferably acne scars, or more preferably acne scars caused by acne cysts or nodules. In preferred embodiments, a p38 inhibitor is administered locally, such as topically, subdermally, or subcutaneously. The p38 inhibitor can be administered independently or in combination with one or more additional agents or treatments. Such agents and/or treatments include, but are not limited to dermabrasion, laser resurfacing, chemical peels, punch techniques, subcision, and augmentation. For example, a p38 inhibitor can be administered prior to, simultaneous with, or after a dermabrasion treatment, laser treatment, chemical peel, punch treatment, subcision and/or augmentation. The amount and frequency of administering the p38 inhibitor and/or additional treatments will depend on various factors (e.g., age of patient, location of acne scar, number of treatment cycles, skin coloration, etc.).

Another example of a skin condition or scar that can be treated by the present invention is a keloid. A keloid is an overgrowth of dense fibrous scar tissue that usually develops after healing at a sight of skin injury. Keloid formation is associated with excessive amounts of collagen, overproduction of which is a skin cell response to injury. A keloid typically grows beyond the boundaries of the original wound, but it rarely extends into the underlying subcutaneous tissue. Keloids are typically is raised and nodular. Keloids can range in their consistency from soft and doughy to rubbery hard. Early keloid lesions are often erythematous. The lesions are first brownish red and later become pale. Lesions are usually devoid of hair follicles and other functioning adnexal glands.

Once a keloid region occurs, its clinical course may vary. Most keloids continue to grow for weeks or months and others for years. Growth is usually slow, but keloids may occasionally enlarge rapidly, tripling in size within months. Once a keloid stops growing, it is usually asymptomatic and remains stable.

Keloids have a high recurrence rate, with over 50% of excised keloids recurring within several years after excision. While keloids are generally a cosmetic concern, they can sometimes cause contractures which may result in a loss of function if they are located over a joint or on the face.

Keloids are more common in people with dark skin complexions. For example, it is estimated that keloids form more frequently in Polynesians and Chinese than in Indians and Malaysians. Moreover, it is estimated that as many as 16% of black Africans have keloids. Whites and albinos are the least affected by keloids. Keloids are also more common in young women than in young males. However, it is believed that this abnormality is related to the fact that more your women pierce their ears than men, causing a physical injury to the skin that may result in a keloid. Keloids occur at a higher rate in individuals aged 10-30 years. Keloids occur less frequently at the extremes of ages, although an increasing number of prestemal keloids have resulted from coronary artery bypass operations and other similar procedures now undertaken in older patients.

It is believed that keloid formation is linked to a genetic component and, therefore, keloid formation tends to run in families. Keloids are thought to be associated genetically with human leukocyte antigen B14, human leukocyte antigen B21, human leukocyte antigen Bw16, human leukocyte antigen Bw35, human leukocyte antigen DR5, human leukocyte antigen DQw3, and blood group A. Transmission is reported as both autosomal dominant and autosomal recessive.

A hypertrophic scar is somewhat similar to a keloid. Like a keloid, it is associated with excessive amounts of collagen overproduction that results from a skin cell's response to injury. However, unlike a keloid, it remains within the boundaries of the original trauma or injury and is typically flat and smooth. Hypertrophic scars have a tendency for spontaneous regression over time.

Treatment of keloids and hypertrophic scars depends upon their location, size, depth, age of the patient, and past response to treatment. Currently treatments include the use of occlusive dressings, compression therapy, intralesional corticosteroid injections, cryosurgery, excision, radiation therapy, laser therapy, interferon therapy, and imiquimod 5% cream (see Berman, B., eMedicine Journal, September 6 (2001) Vol. 2, No. 9, at http://www/arabmedmag.com/issue-31-05-2003/dermatology/main05.htm).

Thus, the present invention involves the prevention and treatment of scars (e.g., keloids and hypertrophic scars) using one or more of the compositions herein containing a p38 inhibitor. A composition containing a p38 inhibitor may be administered independently or in combination with one or more additional agents and/or treatments. Examples of agents and/or treatments that may be useful in a combination treatment include, but are not limited to, occlusive dressings, compression therapy, intralesional corticosteroid injections, cryosurgery, excision, radiation therapy, laser therapy, interferon therapy, and imiquimod. The composition containing the p38 inhibitor is preferably administered locally, e.g., topically, transdermally, or subcutaneously. The p38 inhibitor may be administered prior to, simultaneous with, or after the administration of an additional agent. Preferably, the p38 inhibitor is administered prior to the administration of an additional agent or treatment.

Another common skin and hair disease is scleroderma. Scleroderma is believed to be an autoimmune disease that involves the gradual hardening and tightening of the skin due to excessive collagen production. This results in the “suffocation” of hair follicles, which in turn atrophy. The excess collagen production occurs in patches, which results in hair loss that occurs in distinct areas.

While scleroderma may develop spontaneously, it is believed that it may be induced in people who work with silica, vinyl-chloride, after silicone implants or after injection of certain drugs. Bone marrow transplant recipients and people who contract hepatitis C are also believed to be more likely to develop scleroderma. Scleroderma is three times more common in women than in men. Furthermore, it is believed that at least some of those affected by scleroderma are genetically susceptible to the condition.

The first symptoms of scleroderma often involve a premature graying of the hair followed by hair loss. When hair loss occurs on the scalp, treatment can include surgery to remove the affected skin region.

There are several different classifications of scleroderma that are distinguishable based on the progressive stage of the disease. “Localized scleroderma” refers to a small region of skin affected by scleroderma. Localized scleroderma may often be associated with a patchy hair loss. “CREST,” or “calliopsis (calcium deposits in soft tissue), Reynaud's phenomenon (hypersensitivity of the digits to cold), esophageal involvement (difficulty swallowing), sclerodactyly (skin hardening on fingers), and telangiectasis (dilation of blood vessels around the mouth),” is a more progressive form of scleroderma. While fairly benign, CREST may result in an occasional heart failure. Progressive systemic sclerosis (PSS) is the most progressive form of the disease. PSS is the result of a continued fibrosis in any or all these organs. In PSS, the scleroderma affects internal as well as external parts of the body. For example, joints, gut, lungs, kidneys, nerves and muscles (including those of the heart) may be affected by PSS.

It is believed that the overproduction of collagen which results in scleroderma results from lymphocyte cells that produce cytokines which in turn stimulate fibroblast cells and promote collagen production. In the heart, collagen overproduction and fibrosis can lead to rhythm disturbances and heart failure.

Psoriasis is another skin disease that affects up to 2% of the world's population. Psoriasis is a chronic, immune-mediated, non-contagious disease. It is believed that psoriasis has a genetic component, as Caucasians are the more susceptible to this condition than other ethnic groups. While psoriasis may develop at any age, the most common age for it to begin is in the mid thirties. While the exact cause of psoriasis is still unknown, it has been shown that onset may be preceded by streptococcal infection or stress in some cases.

Clinically, psoriasis often looks like a pink patch of raised skin that is covered in small scales of flaky, white, dead skin. Psoriasis can cause itching and burning sensations. It is believed that in addition to affecting the skin, psoriasis may also causes hair loss. For example, a psoriasis plaque (affected patches of skin) may contain hair follicles that have been forced into the telogen resting stage by the condition. This results in few visible hairs being present in the psoriasis plaques. Thus, telogen effluvium is a typical form of hair loss that affects psoriasis patients. Additionally, psoriasis may sometimes cause a scarring alopecia. While the psoriasis-induced telogen effluvium is fully reversible with proper treatment, the psoriasis-induced scarring alopecia is a permanent form of hair loss. Overall, it is believed that psoriasis is caused by the immune system sending faulty signals which result in a hasten growth cycle in skin cells.

While there are no current cures for psoriasis, some treatments may be useful to control the disease. For example, a tar shampoo may treat a mild case of psoriasis, while a shampoo containing dithranol may be used to treat a more extensive form of the disease. For severe cases, a corticosteroid treatment may be helpful. A corticosteroid treatment can involve topical creams or sometimes local corticosteroid injections into the affected skin area. Recently, preparations containing calcipotroil have been shown to be very useful in treating scalp psoriasis.

Eczema is a chronic skin rash that is extremely itchy. It consists of numerous bumps (papules) or blisters that appear on inflamed, scaly skin. The papules progress into tiny blisters. Scratching of the blisters is often provoked by severe itching and may result in bleeding, ulceration and secondary infections of the affected skin.

Atopic dermatitis is a type of eczema sometimes referred to as infantile eczema or allergic eczema. Atopic dermatitis affects 10% to 12% of all children with symptoms typically appearing within the first few months of a child's life, or before the age 5. Onset of atopic dermatitis after the age of 30 is less common and is often due to exposure of the skin to harsh or wet conditions. Atopic dermatitis often occurs on both sides of the body symmetrically. Atopic dermatitis can cause the skin to become inflamed with redness, swelling, cracking, weeping, crusting and scaling.

Thus, the present invention involves the prevention and treatment of scleroderma, psoriasis, eczema, and atopic dermatitis by administering locally any of the compositions herein. In particular, the present invention contemplates the administration of at least one p38 inhibitor to an affected area topically, transdermally, or subcutaneously. The composition can be administered independently and/or in combination with one or more additional agents or treatments. Examples of agents and/or treatments include, but are not limited to tar, dithranol, a corticosteroid, calcipotroil, and imiquimod.

Vitiligo is another example of a skin condition. Vitiligo results from loss of pigment which produces white patches. Any part of the body may be affected. Usually both sides of the body are affected. Common areas of involvement are the face, lips, hands, arms, legs, and genital areas. Vitiligo affects one or two of every 100 people. About half of those who develop vitiligo, develop the disease before the age of 20. About one-fifth of those who develop vitiligo have a family member with the same condition.

It is believed that vitiligo may be an autoimmune process whereby the body makes antibodies again its own melanocyte pigment cells. Melanocytes make melanin, the pigment that determines color of skin, hair, and eyes. If these cells die or cannot form melanin, the skin becomes lighter or completely white. However, most people with vitiligo are in good general health, although vitiligo may occur with other autoimmune diseases such as thyroid disease.

The degree of pigment loss in vitiligo patients can vary within each vitiligo patch. There may be different shades of pigment in a patch, or a border of darker skin may circle an area of light skin. Vitiligo often begins with a rapid loss of pigment. This may continue until, for unknown reasons, the process stops. Cycles of pigment loss, followed by times where the pigment doesn't change, may continue indefinitely. It is rare for skin pigment in vitiligo patients to return on its own. Some people who believe they no longer have vitiligo actually have lost all their pigment and no longer have patches of contrasting skin color. Although their skin is all one color, they still have vitiligo.

The course and severity of pigment loss differ with each person. Light-skinned people usually notice the contrast between areas of vitiligo and suntanned skin in the summer. Year round, vitiligo is more obvious on people with darker skin. Individuals with severe cases can lose pigment all over the body. There is no way to predict how much pigment an individual will lose. Topical corticosteroids creams containing corticosteroid compounds can be effective in returning pigment to small areas of vitiligo.

PUVA is a form of repigmentation therapy where a type of medication known as psoralen is used. This chemical makes the skin very sensitive to light. Then the skin is treated with a special type of ultraviolet light call UVA. Sometimes, when vitiligo is limited to a few small areas, psoralens can be applied to the vitiligo areas before UVA treatments. Other treatment options include a new topical class of drugs called immunomodulators.

The present invention contemplates a method for preventing or treating vitiligo by administering to a patient susceptible to or suffering from vitiligo an effective amount of a composition that includes at least one p38 inhibitor. The composition is preferably administered locally to a region affected by vitiligo or susceptible to vitil calcipotroil igo. The composition can be administered independently or in combination with one or more other agents. Other agents include, for example, corticosteroids, psoralen, immunomodulators, etc. In some embodiments, the p38 inhibitor will be administered prior to, simultaneous with, or after the administration of an additional agent. Preferably, the p38 inhibitor will be administered prior to the administration of the additional agent (e.g., a corticosteroid or psoralen).

While hair loss itself may not pose a serious health concern, it plays an important social role. Fullness of hair is often associated by society as a manifestation of youthfulness and physical condition. Thus, hair loss may impair an individual's attraction and mating ability.

Furthermore, hair on the scalp provides protection. Primarily, it protects the head from mechanical shock, heat loss, and exposure to ultraviolet (UV) light. Similarly, specialized hairs, such as eyelashes and eyebrows protect the eyes from airborne particles and sun exposure. Moreover, hair in the ear canal and nasal passages helps to filter out particles and pathogens in protecting internal organs.

The loss of hair is often a clinical manifestation of hair disease. Hair loss occurs when the number of hairs lost exceeds the number of hairs regenerated. The average human scalp is covered by approximately 100,000 hair follicles. A hair follicle is a tube-like opening in the epidermis where the hair shaft develops and into which the sebaceous glands open. Normally, roughly 50-100 hairs randomly fall out a day. This is unnoticeable because the lost hair is replaced by as new hairs daily, as each hair follicle undergoes a hair cycle.

Hair goes through a characteristic cycle consisting of an immature phase, a growing phase called anagen, a transitional phase between the growing phase and the resting phase called catagen, and finally a resting phase called telogen in which the hair stops growing awaiting to fall out. At any given time, 85 to 90% of hairs on our body are in anagen phase or growing phase, which lasts anywhere from two to five years. This phase is followed by a short regression phase, or catagen, which lasts 2-3 weeks. Approximately 1% of hair follicles are in catagen. Approximately 10-15% of hair follicles are in the resting phase, the telogen, which lasts about 3-5 months. A hair follicle typically goes through 10-20 asynchronous cycles during its lifetime. Persistent loss of more than 100 hairs, more preferably more than 150 hairs a day, more preferably more than 200 hairs a day, more preferably more than 300 hairs a day, or more preferably more than 400 hairs a day would consist a state of hair loss, or alopecia, albeit it could be temporary.

Hair conditions that leads to hair loss is often mediated by the immune system and is often associated with inflammation of the hair follicle. Examples of hair diseases that are mediated by the immune system include, but are not limited to, alopecia areata, alopecia cicatrisata, alopecia totalis, alopecia universalis, alopecia keratosis pilaris, alopecia triangularis, anagen effluvium, androgeneic alopecia, androgenetic alopecia, area celsi, bacterial follicultiis, black piedra, blackdot ringworm, cemical alopecia, cicatrical alopecia, chronic telogen effluvium, dermatophyte infection, diet deficiency induced alopecia, diffuse alopecia, dissecting cellulites, drug induced alopecia, eosinophilic pustular folliculitis, erosive pustular dermatosis, familial focal alopecia, feldman syndrome, female alopecia, female pattern baldness, follicular degeneration syndrome, folliculitis barbae, folliculitis decalvans, folliculitis keloidalis, graham-little syndrome, herpes simplex folliculitis, herpes zoster folliculitis, hot comb alopecia, involutional alopecia, ischemic alopecia, keratosis follicularis spinulosa decalvans cum ophiasi, lichen planopilaris, lipedematous alopecia, loose anagen syndrome, loose hair syndrome, male pattern baldness, mechanically induced alopecia, mixed inflammatory alopecia, non-scarring alopecia, occipital alopecia, occipital alopecia areata, ofuji syndrome, papular atrichia, pattern baldness, perifolliculitis capitis abscedens et suffodiens of hoffman, perinevoid alopecia areata, postpartum alopecia, pseudofolliculitis barbae, pseudopelade of brocq, ringworm, sarcoidosis, scarring alopecia, telogen effluvium, thermal alopecia, tick bite induced alopecia, tinea capitis, traction alopecia, traction folliculitis, traumatic alopecia, triangular alopecia, trichomycosis axillaries, trichotillomania, tufted hair folliculitis, and vaccination induced alopecia.

Alopecia is a condition of excessive, premature hair loss. Alopecia may be caused by many factors. These facts include, but are not limited to, genetic factors, agin, or local or systemic disease.

In alopecia areata, a patient experiences a sudden loss of hair in circumscribed areas. Such patients have no obvious skin disorder or systemic disease. Any hairy area may be involved in alopecia areata. The scalp and beard are most commonly affected by alopecia areata. In some cases, such as alopecia universalis, all body hair is lost.

In female alopecia, a female patient experiences a loss of hair. Female alopecia is usually the result of genetic factors. Female alopecia is thought to be associated with hormones and an increase in male testosterone hormone. Hormone changes that occur as a result of childbirth, contraceptive pills, anemia, and menopause, for example, can cause female alopecia. It is thought that female alopecia is caused by a dominant gene that must be present in both parents which is passed down to a daughter.

Current treatments for hair loss include, but are not limited to, Minoxidil (e.g., 5% conc.), laser phototherapy, Revivogen, Toppe™, and Shen Min™. Minoxidil is a hair growth product that specifically works on the hair follicles, which have miniaturized due to male or female pattern baldness. Minoxidil forces the hair follicles to go into the growth phase. Although Minoxidil is a vasodilator its effects are not contributed to its ability to increase circulation and its exact mode of action remains unknown. Laser phototherapy is a new treatment that is believed to stimulate hair growth. Laser phototherapy can be applied using, for example, the LaserComb™. Another form of hair growth treatment is Revivogen™. Revivogen™is a recently approved drug that blocks the enzyme, 5-alpha-reductase. Typically, 5-alpha-reductase enzyme helps generate a hormone known as dihydro-testosterone (DHT). DHT is associated with the loss of functioning in the hair follicle. Toppek™, another new system to prevent hair loss, functions by opening the hair shaft and allowing the infusion of keratin protein into the hair shaft. Furthermore, Shen Min™, a 100% natural hair nutrient, which is derived from the eastern wild rose He Shou Wu is also thought to help generate new hair growth and restore hair color. Thus, any of the above treatments (or any other known treatments) can be used in combination with the compositions herein.

Thus, the present invention relates to methods of preventing and/or treating hair loss or hair conditions, e.g., alopecia arearta and female alopecia. In particular, the present invention contemplates a method for preventing and/or treating a hair condition in a patient by administering to such a patient an effective amount of at least one p38 inhibitor. The administration of such compound is preferably made locally, e.g., topically, subcutaneously, transdermally. The administration of the p38 inhibitor(s) can be accompanied with one or more other agents (e.g., Minozidil or Revivogen) or treatments (e.g., laser photo therapy). The p38 inhibitor can be administered prior to, simultaneous with, or after the administration of the additional agent. In preferred embodiments, the p38 inhibitor is administered prior to the administration of other agents.

Topical applications to the skin or a mucous membrane using a cream, lotion, gel, oil, ointment, suspension, aerosol spray, powder, semi-solid formulation (e.g., a suppository), or article of manufacture, all formulated so as to contain a therapeutic composition of the present invention using methods well-known in the art.

As used herein an “effective amount” refers to the amount of a composition, which produces a desired outcome. For example, an “effective amount” for a therapeutic use is an amount of a composition comprising an active compound (e.g., a p38 inhibitor) that is required to provide a clinically significant increase in preventing or treating a conditions, e.g., stimulating and/or augmenting hair growth, reducing and/or eliminating vitiligo patches, etc.

The present invention also contemplates combination therapies (e.g., treatments using two or more p38 inhibitors or a combination of a p38 inhibitor and another agent). In cases of combination therapy, a synergistic effect may result such that the effect achieved with the combination of methods and compositions of this invention is greater than the sum of their effective amounts independently. Thus, the present invention contemplates a synergistic effect may occur when administering two or more p38 inhibitors or when administering a p38 inhibitor and another agent.

In some embodiments, the compositions herein are administered in about one to 100 applications, preferably about one to 50 applications, more preferably about one to 25 applications, or more preferably about one to 10 applications.

Each application of the compositions herein generally consists of about 1 mg to 100 g concentration of a p38 inhibitor per application, more preferably about 10 mg to 10 g concentration of a p38 inhibitor per application, or more preferably about 50 mg to 1 g concentration of a p38 inhibitor per application. In some embodiments, a daily dose consists of about 0.01 mg/kg body weight to 100 mg/kg body weight, preferably between about 0.1 mg/kg body weight and about 50 mg/kg body weight, and more preferably between about 0.5 mg/kg body weight to 30 mg/kg body weight.

Application(s) are preferably administered for a period of about one day and up to about one year. However, longer or lifelong treatments are also contemplated, especially for preventative treatments. In preferred embodiments, applications are administered about once every twelve hours and up to about once every month. Preferably, two to four applications of the therapeutic composition are administered per month, or more preferably two to four application of the therapeutic composition are administered per week, or more preferably two to four application of the therapeutic compositions are administered per day.

For topical applications, the compositions herein are preferably applied to targeted area daily, bi-weekly, weekly, or at other regular intervals. The specific route, dosage, and timing of the administration will depend, in part, on factors, including but not limited to, the age, weight, sex, and medical condition. Topical formulations can be applied as a topical gel, spray, ointment or cream containing the active ingredients (including a p38 inhibitor) in a total amount of, for example, 0.075 to 90% w/w, preferably 0.2 to 50% w/with, and most preferably 0.4 to 25% w/w.

A transdermal device can also be used to administer the compositions of the present invention. Preferably, topical administration is accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent can also function as the membrane. The transdermal patch can include the compound in a suitable solvent system with an adhesive system, such as an acrylic emulsion, arid a polyester patch.

The present invention and methods herein also contemplates changing skin coloration.

Changes in skin coloration can be caused by numerous biological and non-biologic factors. Non-biologic factors that can cause alterations in skin colorations include tattoos. The word tattoo comes from the Tahitian “tatu” which means “to mark something”. It is arguably claimed that tattooing has existed since 12,000 years BC. Three examples of tattoos include: decorative tattoos, traumatic tattoos and gunpowder tattoos. Decorative tattoos are made by repeatedly puncturing of the skin with a needle saturated with colored ink. Traumatic tattoos can occur, for example, if the skin is grazed along the surface of a road and tiny pieces of grit and carbon powder enter the skin. Gunpowder explosions can cause tattooing if the gunpowder penetrates the skin.

Today, decorative tattooing is very common. It is approximated that over 10 million Americans have at least one tattoo, and that close to 4,000 tattoo studios currently operate in the United States. Many people use tattoos to alter skin coloration for aesthetic and cosmetic reasons. For example, some individuals tattoo permanent makeup (e.g., on eyelids, lips, eyebrows, etc.) to save time or because they have physical difficulty applying regular, temporary makeup. Tattooing can also be an addition or substitution to reconstructive surgery, particularly of the face or breast, to simulate natural pigmentation. In some instances, people who have lost their eyebrows due to alopecia (a form of hair loss) may choose to have “eyebrows” tattooed on, while others with vitiligo (a lack of pigmentation in areas of the skin) may try tattooing to help camouflage the condition. Furthermore, tattooing can be part of an initiation right (e.g., to a fraternity or a gang).

Tattooing involves rapidly and repeatedly injecting ink into the dermal layer of the skin with a small needle to develop a permanent coloration. A small tattoo tales about 45 minutes and a larger one may take many hours or repeated visits. The inks used by most tattoo artists are not really inks but rather pigments that are suspended in a carrier solution. The pigments are usually not vegetable dyes. Instead, today's pigments are primarily metal salts. However, some pigments are plastics and there are some vegetable dyes that are used as well. The pigment provides the color of the tattoo. The purpose of the carrier is to disinfect the pigment suspension, keep it evenly mixed, and provide for ease of application.

The pigment, grit, carbon or ink used for tattooing is considered a food additive by the Food and Drug Administration (FDA) and causes minimal adverse reactions. Under a microscope, tattoos appear as tiny granules of color pigment. Tattoo granules are initially dispersed in the upper dermis and vertical foci at sites of injection. Approximately 7-14 days after injection, the granules concentrate at a more focal location. Tattoo granules are composed of loosely packed particles, ranging from approximately 2-400 nm in diameter. The most common particle size is about 40 nm. Less common particle sizes are about 2-4 nm is size and about 350-400 nm is size.

Tattoo granules are endocytosed by fibroblasts as well as macrophages in the dermis and subcutis. Normally, foreign bodies are attacked and removed from the body by the natural defense mechanism of macrophage activity. However, tattoo particles are sufficiently large to inhibit activity by macrophages and tattoo pigment, grit, carbon or ink remains in the skin. This results in an appearance of macrophage “freezing.” See Fujita H, Arch. Histol. Cytol. (1988) Jul; 51(3):285-94. Thus, a tattoo is relatively permanent.

The oldest pigments came from using ground up minerals and carbon black. Today's pigments include the original mineral pigments, modern industrial organic pigments, a few vegetable-based pigments, and some plastic-based pigments. Allergic reactions, scarring, phototoxic reactions (i.e., reaction from exposure to light, especially sunlight), and other adverse effects are possible with many pigments. The plastic-based pigments are very intensely colored, but there are many reported adverse reactions to them. Recently, there has been development of pigments that glow in the dark or in response to black (ultraviolet) light. While some of these pigments may be safe, others are radioactive or otherwise toxic. Below is a table listing some commonly used pigments in tattoo inks. This list is not exhaustive. Just about anything can be used as a pigment. Also, many inks mix one or more pigment:

TABLE 1
Commonly used compositions in tattoo inks

| Final Color | Material Used |
|---|---|
| Black | Iron Oxide (Fe₃O₄); Iron Oxide (FeO); Carbon; Logwood |
| Brown | Ochre |
| Red | Cinnabar (HgS); Cadmium Red (CdSe); Iron Oxide (Fe2 O3); Napthol-AS |
| | pigment |
| Orange | disazodiarylide and/or disazopyrazolone; cadmium seleno-sulfide |
| Flesh | Ochres (iron oxides mixed with clay) |
| Yellow | Cadmium Yellow (edS, CdZnS); Ochres; Curcuma Yellow; Chrome |
| | Yellow (PbCrO4, often mixed with PbS); disazodiarylide |
| Green | Chromium Oxide (Cr₂O₃), called Casalis Green or Anadomis Green; |
| | Malachite [Cu₂(CO₃)(OH)₂]; Ferrocyanides and Ferricyamides; Lead |
| | chromate; Monoazo pigment; Cu/Al phthalocyanine; Cu phthalocyanine |
| Blue | Azure Blue; Cobalt Blue; Cu-phthalocyanine |
| Violet | Manganese Violet (manganese ammonium pyrophosphate) ; Various |
| | aluminum salts; Quinacridone; and Dioxazine/carbazole |
| White | Lead White (Lead Carbonate); and Titanium dioxide (TiO₂) |
| | Barium Sulfate (BaSO₄); and Zinc Oxide |

Until recently, government has not attempted to regulate the use of tattoo inks and the pigments used in them. However, with the growing popularity of tattooing and permanent makeup, the U.S. federal drug agency has begun looking at safety issues concerning tattoo removal, adverse reactions to tattoo colors, and infections that result from tattooing.

Beyond the pain often associated with getting a tattoo, there are numerous risks involving both tattooing and removal of a tattoo. These risks include infection, allergic reactions, granulomas, keloid formation, MRI complications and removal problems. Infection is common and can be avoided by using clean needles and sterile ink. Allergic reactions to tattoo pigments are rare. However, when they do occur they may be particularly troublesome, especially because the pigments may be hard to remove. Thus, it may be desirable to remove a tattoo due to an allergic reaction to the pigment or ink. Granulomas are nodules that may form around material such as tattoo ink that the body perceives as foreign. If and when a granuloma is formed, it may be desirable to quickly remove to the tattoo. Keloid formation are scars that grow beyond normal boundaries. Keloids may form from an injury or trauma to the skin. Tattooing (and tattoo removal) can cause keloid formations especially in individuals who are susceptible to such formations. Additional complication associated with tattoos include reports that people with tattoos or permanent makeup who experienced swelling or burning in the tattooed areas when they undergo magnetic resonance imaging (MRI). This seems to occur only rarely and apparently without lasting effects. However, there are also reports that tattoo pigments can interfere with the quality of the image. This seems to occur mainly when a person with permanent eyeliner undergoes MRI of the eyes.

The most common reason people with tattoos seek medical care is that they want the tattoo removed. Conservative estimates suggest that almost 50 percent of all people who get tattoos later decide to remove them. Despite advances in laser technology, tattoo removal is a painful process that usually involves multiple treatments and a considerable expense. Complete removal without scarring may be impossible. Currently, there are several methods for tattoo removal. The most popular of these methods include: excision, dermabrasion, laser therapy, cryosurgery, grafting, camouflaging, scarification, and salabrasion.

Excision involves an injection of a local anesthetic to numb the area after which the tattoo is removed surgically. The edges are then brought together and sutured. With this procedure, there is minimal bleeding which is easily controlled with electrocautery. In some cases involving large tattoos, a skin graft taken from another part of the body may be necessary. Excision sometimes involves the use of tissue expanders (balloons inserted under the skin, so that when the tattoo is cut away, there is less scarring). Larger tattoos may require repeated surgery for complete removal.

Dermabrasion, which is usually used for smaller tattoos, involves spraying the tattoo with a solution that freezes the area. The tattoo is then “sanded” with a rotary abrasive instrument causing the skin to peel. Because some bleeding is likely to occur, a dressing is immediately applied to the area.

Laser therapy is a popular technique for tattoo removal. Commonly used lasers include the Versapuls C with helper H laser, Q-switched Nd:YAG (532 nm, 1064 nm), Q-switched alexandrite (855 nm), and the Q-switched ruby (694 nm). Recent developments in laser therapy involve the development of picosecond lasers. The present invention contemplates all other lasers. Q-switched ruby and alexandrite lasers are useful for removing black, blue, and green pigments. The Q-switched 532 nm Nd:YAG laser can be used to remove red pigments and the 1064 Nd:YAG laser is used to remove black and blue pigments. Thus, often time more than one wavelength or laser is used to remove a multi-colored tattoo. After the tattoo area is numbed, pulses of light from a laser are directed onto the tattoo. The laser breaks up the tattoo pigment, and subsequently, the body's scavenger cells remove the treated pigmented areas. Generally, several visits are necessary over a span or weeks or months, and the treatments can be expensive. Some individuals experience hypopigmentation—a lightening of the natural skin coloring—in the affected area. Laser treatments also can cause some tattoo pigments to change to a less desirable shade.

Cryosurgery is the freezing of tissue prior to its removal or excision. Grafting involves removing a skin graft taken from another part of the body to cover a tattooed region. Scarification involves removing the tattoo with an acid solution and creating a scar in its place. Camouflaging a tattoo entails the injection of new pigments either to form a new pattern or cover a tattoo with skin-toned pigments. However, it is noted that injected pigments may not to appear natural because they lack the skin's natural translucence.

Salabrasion is a procedure similar to dermabrasion in which the tattooed area is first numbed with a local anesthesia. Subsequently, a solution of ordinary tap water dipped in table salt is applied to the area, and an abrading apparatus such as the one used with dermabrasion, or an even simpler device such as a wooden block wrapped in gauze, is used to vigorously abrade the area. When the area becomes deep red in color, a dressing is applied.

Regardless of which technique is used, tattoo removal generally results in textual changes, scarring, and discoloration. In rare cases, localized and generalized allergic reaction can occur. The effectiveness of tattoo removal depends on various factors, including but not limited to, the size of the tattoo, the location of the tattoo, the individual's healing process, how the tattoo was applied, and the length of time that the tattoo has been on the skin. A tattoo performed by a more experienced tattoo artist, for example, may be easier to remove since the pigment is evenly injected in the same level of the skin. A tattoo that has been on the skin for a considerable length of time may be more difficult to remove than a new one.

Preliminary results from a recent animal study suggest that topical imiquimod 5% cream used in the acute phase after tattooing may have utility as a nonsurgical method for pigment removal. See Dermatol. Surg., 28(1) (2002); see also Derm. Times, 22(4) (2001), both of which are incorporated herein by reference for all purposes. This study involved five albino guinea pigs that were tattooed with black, red, green and yellow dye. A punch biopsy was taken with 6 hours after tattooing. Then one animal served as control and the others were allocated to one of four treatments: petrolatum, tretinoin 0.025 percent, imiquimod 5 percent cream, and tretinoin alternated with imiquimod. Each agent was applied every 6 hours for seven days, and the responses were evaluated clinically, and with repeat biopsies at seven and 28 days after tattoo placement. Macroscopically and histologically, imiquimod alone appeared to be the most effective regimen for fading the tattoo. However, the biopsy evaluation also revealed the presence of epidermal and dermal necrosis with separation, severe inflammation and fibrosis, and disruption of the skin appendages at the imiquimod-treated site.

Imiquimod is a small molecule, which is a toll-like receptor (TLR) agonist that is capable of indirectly activating multiple arms of the innate immune response. FIG. 1 illustrates the indirect activation of the immune system by imiquimod. In particular, imiquimod binds TLR-7 on the cell surface and generates a signal via the TRAF6 pathway. This signaling pathway leads to the nucleus of the cell via the p38 , JNK1, or NF-KB MAP kinase pathways. Activation of the above signaling pathways induces the production of pro-inflammatory cytokines, including but not limited to TNF-α, Interferon-α, and IL-1.

Thus, the present invention contemplates the local and direct administration of cytokines as a means for altering skin coloration. The term “cytokine” as used herein refers to any substance produced by cells that has a specific effect on cell-cell interaction, communication and/or behavior of other cells. More preferably, a cytokine is any substance released by cells that has a specific effect on cell-cell interaction, communication and/or behavior of other cells. In some embodiments, a cytokine is a small protein or a biological factor. Preferably, a cytokine is in the range of 1-40 kD, more preferably 2-30 kD, more preferably 3-20 kD, or more preferably 4-25 kD. In preferred embodiments, a cytokine is selected from the group consisting of interleukins, lymphokines, tumor necrosis factors, interferons, chemokines, and growth factors.

Interleukins are secretory proteins produced by lymphocytes, monocytes and other cells types. Interleukins are often released by cells in response to antigenic and non-antigenic stimuli. Examples of interleukins include, but are not limited to, IL-1 through IL-15. In preferred embodiments, a cytokine of the present invention is IL-1 or IL-2, or any homologs, derivatives, variants, or mimetics thereof. More preferably, a cytokine of the present invention is IL-1, or any homologs, derivatives, variants, or mimetics thereof.

Lymphokines are soluble factors that are secreted by activated lymphocytes and that affect other lymphocytes and other cell types. Representative examples of lymphokines include, but are not limited to, IL-1 through IL-15, GM-CSF, G-CSF, M-CSF, alpha.-, beta.-, or gamma-interferon, tumor necrosis factors, and their respective receptors. In preferred embodiments, a lymphokine is selected from the group consisting of a CSF receptor, alpha-interferon, interleukins-2 or any homologs, derivatives, variants, or mimetics thereof. More preferably, a lymphokine is interferon- a, or any homologs, derivatives, variants, or mimetics thereof.

Tumor necrosis factors are cytokines produced mainly by macrophages and T lymphocytes that help regulate the immune response and hematopoiesis (blood cell formation). Examples of tumor necrosis factors include: TNF-α (also called cachectin) and TNF-β (also called lymphotoxin). TNF-α is produced by macrophages, while TNF-β is produced by activated CD4+T cells. In preferred embodiments, a cytokine of the present invention is TNF-α or any homologs, derivatives, variants, or mimetics thereof.

Interferons are glycoproteins derived from human cells that normally play a role in fighting viral infections by preventing virus multiplication in cells. There are multiple types of interferons (e.g., Type I and Type II). Examples of interferon Type I cytokines include, but are not limited to, interferon-α and interferon-β. Examples of interferon Type II cytokines include, but are not limited to, interferon-γ. Preferably, a cytokine of the present invention is interferon-a or any homolog, derivative, variant, or mimetic thereof.

Chemokines are cytokines that are chemotactic for leucocytes. Chemokines can be subdivided into two general groups on the basis of the arrangement of a pair of conserved cysteines: the C×C group includes platelet Factor 4, platelet basic protein, IL-8, melanoma growth stimulatory protein, and macrophage inflammatory protein 2. The C C group, on the other hand, include, but are not limited to, TECK, TARC, RANTES, MIP-1, MCP-1, MCP-3, MCP-4, MDS, MIP-1, MIP-3, MIP-4, Eotaxin-1, Eotaxin-2, and Exodus-1.

Growth factors are substances produced by a leucocyte that acts upon another cell. Examples are interleukins, interferon-alpha, lymphotoxin, tumor necrosis factors, erythropoietin (epoietin-α), and colony-stimulating factors (CSFs). Colony-stimulating factors stimulate production of white blood cells (WBCs). Examples of CSFs include, but are not limited to, granulocyte-CSF (C-CSF) (e.g., filgrastin), and granulocyte macrophage-CSF (GM-CSF) (e.g., sargramostim). Examples of commercial embodiments of CSFs include, but are not limited to, Leukine™, Neupogen™ and Neulasta™. Each of the above CSFs varies slightly in its effect on the body and in the indications for which they are marketed for usage. In preferred embodiments, the cytokine of the present invention is a growth factor but not a CSF. In other embodiments, the cytokine of the present invention is a CSF selected from the group consisting of Leukine™, Neupogenm and Neulasta™.

In addition to cytokines, the present invention also contemplates the use of substances that stimulate or enhance cytokine production. Examples of substances that stimulate or enhance cytokine production include, but are not limited to, flagellum (stimulating CSFs), Echinacea, endothelins, vitamin A, vitamin B5, anti-oxidants, etc.

In particular the present invention contemplates the local administration of one or more substances (cytokine or substance that induces or enhances cytokine production) to alter skin coloration. Such substances are preferably administered to a dermal region desirable of being of a different color. In some embodiments, the dermal region includes a tattooed region. The tattoo can be, for example, a decorative tattoo, a traumatic tattoo, or a gunpowder tattoo, and it may be desirous to either change the coloration of the tattoo or to remove or reduce the coloration from the tattoo.

In one example, a dermal skin region desirable of being of a different color is a decorative tattoo having one of more pigments. The pigments can be any one of the pigments disclosed herein or any other pigments, whether or not approved for tattoo use.

The methods for altering skin coloration disclosed herein include administering, preferably locally, to the dermal skin region desirable of being of a different skin color one or more of the compounds disclosed herein. In preferred embodiments, a compound administered is a cytokine. More preferably, a compound administered is an interleukin, an interferon, or a tumor necrosis factor. More preferably, a compound administered is IL-1, INF-α, or TNF-α.

When the methods are used to remove or reduce a tattoo, administration of any of the compound herein can occur, for example, immediately after injection of a pigment into the skin (e.g., a mistake by a tattoo artist) or after a prolonged period (e.g., due to an individual's desire to have the tattoo removed). This approach, because it selectively activates only a single art of the immune system, may have fewer side effects and thus better safety to efficacy performance than direct imiquimod application (which activates multiple arms of the innate immune response).

The compounds of the present invention can be part of a kit, which can include one or more cytokines, individually packaged. A kit for skin color alteration would typically comprise at least one compound such as a cytokine or a substance that enhances cytokine production. Preferably, the kit will also contain instructions for proper use and disposal of the contents after use. The instruction can include, for example, a description as to which compound should be used to achieve a particular result (e.g., color alteration) and how to administer the compound.

The compounds of the present invention can be administered by any suitable route, preferably in the form of a pharmaceutical composition adapted to such a route, and in a dose effective for the treatment intended. The active compounds and composition can, for example, be administered orally, intravascularly (IV), intraperitoneally, subcutaneously, intramuscularly (IM) or topically including by way of a patch. In preferred embodiments, the active compounds of the present invention are administered topically to the tattooed region.

The compounds of the present invention can also be administered by injection (IV, IM, subcutaneous or jet) as a composition wherein, for example, saline, dextrose, or water can be used as a suitable carrier. The pH value of the composition can be adjusted, if necessary, with suitable acid, base, or buffer. Suitable bulking, dispersing, wetting or suspending agents, including mannitol and PEG 400, can also be included in the composition. A suitable parenteral composition can also include a compound formulated as a sterile solid substance, including lyophilized powder, in injection vials. Aqueous solution can be added to dissolve the compound prior to injection.

A pharmaceutical composition can contain any compound disclosed herein at any thereapeutically effective amount. Preferably a pharmaceutical composition contains about 0.1 to 1000 mg of a compound (e.g., a cytokine or a substance that enhances cytokine production), more preferably at about 7.0 to 350 mg of a compound, more preferably about 15 to 250 mg of a compound, or more preferably about 20 to 150 mg of a compound. The compounds herein can be administered once per treatment cycle or multiple times per treatment cycle. For example, single or multiple doses can be made prior to, during, or after each color alteration treatment.

In some embodiments, a topical preparation of the compounds herein are applied to the tattooed area 1-10 times a day, more preferably 1-5 times a day, or more preferably 1-3 times a day, and are preferably applied as a topical gel, spray, ointment or cream containing the active ingredients in a total amount of, for example, 0.075 to 30% w/w, preferably 0.2 to 20% w/w and most preferably 0.4 to 15% w/w.

The compounds can be applied prior to, during, or post a color alteration treatment. More preferably, the compounds are applied prior to a color alteration treatment. A color alteration treatment is any procedure (whether chemical, physical, biological, etc.) known by a person of ordinary skill in the art that is used to reduce, alter, or eliminate skin coloration, whether such skin coloration is naturally occurring (e.g., freckles) or non-naturally occurring (e.g., a tattoo). Examples of color alteration treatments include, but are not limited to, excision, dermabrasion, laser therapy, cryosurgery, grafting, camouflaging, scarification, and salabrasion. In preferred embodiments, the color alteration treatment is laser therapy. The compounds herein can be administered prior to, during, and/or post a color (e.g., tattoo) alteration treatment.

In one embodiment, coloration resulting from a tattoo is wholly or partially removed by administering one or more of the compounds disclosed herein to the tattooed dermal region. Such compounds are preferably administered locally, (e.g., topically or transdermally). The compounds are preferably administered prior to or during a color alteration treatment, wherein the color alteration treatment is preferably laser therapy.

When formulated as an ointment, the active ingredients (cytokines) can be employed, for example, with either paraffinic or a water miscible ointment base. Alternatively, the active ingredients can be formulated in a cream with an oil-in-water cream base. If desired, the aqueous phase of the cream base can include, for example at least 30% w/w of a polyhydric alcohol such as propylene glycol, butane-1,3-diol, mannitol, sorbitol, glycerol, polyethylene glycol and mixtures thereof.

The topical formulation can desirably include a compound that enhances absorption or penetration of the active ingredient through the skin or other affected areas. Examples of such dermal penetration enhancers include dimethylsulfoxide and related analogs.

The compounds of this invention can also be administered by a transdermal device. Preferably, topical administration is accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent can also function as the membrane. The transdermal patch can include the compound in a suitable solvent system with an adhesive system, such as an acrylic emulsion, and a polyester patch.

The effective amount of compounds administered and doses will vary depending on the patient's natural skin color, coloration desirous of being removed, added or altered, size of target region desirable of having a different coloration, the location of the target region, and the color alteration treatment used in conjunction with the cytokines.

Additional methods and compositions for treating conditions involve modulating the nervous system and its activity. The nervous system coordinates movements of the body and cellular activities. Most neurons achieve their effect by releasing chemicals, such as neurotransmitters. Neurotransmitters are released from the axon terminal of one neuron, and pass a junction known as the synapse, before reaching a receiving cell (a postsynaptic cell). A postsynaptic cell can be, for example, another neuron, a muscle cell, or a gland cell. Neurotransmitters at excitatory synapses depolarize a postsynaptic cell membrane.

A neurotransmitter that is commonly used throughout the body is Acetylcholine (ACh). Acetylcholine is known to activate two types of receptors, muscarinic and nicotinic receptors. The muscarinic receptors are found in all effector cells stimulated by the postganglionic neurons of the parasympathetic nervous system, as well as in those stimulated by the postganglionic cholinergic neurons of the sympathetic nervous system. The nicotinic receptors are found in the synapses between the preganglionic and postganglionic neurons of both the sympathetic and parasympathetic. The nicotinic receptors are also present in many membranes of skeletal muscle fibers at the neuromuscular junction.

Acetylcholine is released from cholinergic neurons when intracellular vesicles fuse with the presynaptic neuronal cell membrane. Vesicles are generally about 50 nm in diameter and contain about 10,000 molecules of ACh. Vesicle precursors are made in the endoplasmic reticulum (ER) and golgi of the neuronal soma and are transported down the axon to the terminal where the membrane pinches off to create new vesicles.

It is postulated that when ACh binds to its receptors on the postsynaptic cell membrane ligand-gated sodium channels opens up. These ligand-gated sodium channels allow an influx of Na+ ions, which in turn reduces the membrane potential of the postsynaptic cell to an excitatory postsynaptic potential (EPSP). If depolarization of the postsynaptic membrane reaches a particular threshold, an action potential is generated in the postsynaptic cell.

Defects in synaptic vesicle release and/or recycling can cause severe neurological and neuromuscular disorders. Such disorders include, but are not limited to, myasthenic syndromes such as Lambert-Eaton myasthenic syndrome (LEMS), Congenital myasthenic syndrome, botulism, and tetanus toxicity. Defects in synaptic vesicle release and/or recycling can be effectuated by neurotoxins, especially the neurotoxins. In particular, the present invention contemplates the administration of neurotoxins for the inhibition, delay, interference, or decrease of vesicle release and/or recycling.

The present invention relates to compositions and methods for improving neurotoxin treatment, e.g., by increasing the duration of the effect of a neurotoxin. Such compositions and methods are useful in the treatment and prevention of a condition that is treatable or preventable by a neurotoxin.

The term “neurotoxin” refers to any substance that inhibits neuronal function. Neurotoxins are often extremely toxic if taken or applied inappropriately. Neurotoxins can function, for example, against sodium channels (e.g., tetrodotoxin) or by blocking synaptic transmission (e.g., curare and bungarotoxin, botulinum toxin).

Examples of neurotoxins include, but are not limited to, curare, bungarotoxin, saxitoxin, tetrodotoxin, tetanus toxin, and botulinum toxins. Curare neurotoxins are alkaloids that are the active ingredients of arrow poisons used by South American Indians. Curare alkoids have muscle relaxant properties because they block motor end plate transmission, acting as competitive antagonists for acetylcholine. Bungarotoxin is a neurotoxic protein derived from the venom of an elapid snake known as bungarus multicinctus. Alpha-bungarotoxin blocks nicotinic acetylcholine receptors, while beta- and gamma-bungarotoxins act presynaptically causing acetylcholine release and depletion. Saxitoxin is a neurotoxin produced by the red tide dinoflagellates, Gonyaulax catenella and G. Tamarensis. Saxitoxin binds to sodium channels, thus blocking the passage of action potentials. This toxin was originally isolated from the clam, Saxidomus giganteus. Tetrodotoxin is a neurotoxin derived from the Japanese puffer fish. Tetrodotoxin also binds to sodium channel, and its activity somewhat resembles that of saxitoxin. Tetanus toxin is a neurotoxin caused by the anaerobic, spore-forming bacillus Clostridium tetani. Clostridium tetani usually enters the body through contaminated puncture wounds although it may also enter through burns, surgical wounds, cutaneous ulcers, injection sites etc. Tetanus toxicity is often accompanied with sustained muscular contraction caused by repetitive nerve stimulation. Botulinum neurotoxins are produced by the anaerobic, gram-positive bacterium Clostridium botulinum (referred to herein as C. botulinum). Botulinum toxins can cause neuroparalysis, or botulism, in mammals. There are at least seven known types of botulinum toxins: toxins A, B, C₁ (referred to herein as “C”), D, E, F, and G.

The molecular weight of each one of the above seven types of botulinum toxin is about 150 kD. When these botulinum toxins are released by C. bacterium, they are complexed with non-toxin proteins. For example, botulinum toxin type A complex can be produced by Clostridial bacterium as either a 900 kD, 500 kD, or a 300 kD form. Botulinum toxin type B and C are usually produced as a 500 kD complex. Botulinum toxin type D is usually produced as either a 300 kD or a 500 kD complex. Finally, botulinum toxin types E and F are usually produced as an 300 kD complexes.

These complexes of molecular weight greater than about 150 kD are believed to contain a non-toxin hemaglutinin protein and a non-toxin and non-toxic nonhemaglutinin protein. These two non-toxin proteins may act to provide stability against denaturation to the botulinum toxin molecule and protection against digestive acids when toxin is ingested. Additionally, it is possible that the larger botulinum toxin complexes may result in a slower rate of diffusion of the botulinum toxin away from a site of intramuscular injection of a botulinum toxin complex.

While each one of the botulinum neurotoxins has different properties and actions, there are some general structural and functional similarities among all seven botulinum toxins. For example, all seven toxins are synthesized as single-chain polypeptides with molecular weights of approximately 150-kD. These single-chain molecules are activated by proteolytic enzymes by nicking or cleaving. Once it is nicked or cleaved, the 150-kD single-chain molecule forms a dichain molecule consisting of a 100-kD heavy chain (H chain) and a ˜50-kD light chain (L chain) linked by a disulfide bond. The H chain is responsible for high-affinity docking of the neurotoxin to the presynaptic nerve terminal receptor, which enables the internalization of the neurotoxin into the cell. The L chain is a zinc-dependent endopeptidase that cleaves membrane proteins (e.g., SNAP-25 or VAMP) that are responsible for docking neurotransmitter vesicles (e.g., ACh vesicles) on the inner side of the nerve terminal membrane.

Thus, the molecular mechanism by which all of the botulism neurotoxins function can be summarized by the following three steps. In the first step, the neurotoxin binds to the presynaptic membrane of the target neuron through a specific interaction between the H chain and a cell surface receptor. The receptor for each type of botulinum neurotoxin and for tetanus neurotoxin is different. The carboxyl end segment of the H chain (H_{c}) appears to be important for targeting of the toxin to the cell surface. In the second step, the neurotoxin crosses the plasma membrane of the presynaptic cell. The neurotoxin enters the cell through receptor-mediated endocytosis. An endosome containing the neurotoxin is formed. Each endosomes contains a proton pump that decreases the pH inside the endosome. This reduced pH triggers a conformational change within the neurotoxin, which allows it to escape the endosome into the cytoplasm of the presynaptic cell. During the third phase, the disulfide bond joining the H and L chains is reduced. The L chain, which is a zinc (Zn++) endopeptidase, the selectively cleaves SNARE proteins. SNARE proteins, which include syntaxin, VAMP, and SNAP-25 are essential for recognition, docking, release and recycling of neurotransmitter-containing vesicles.

Each neurotoxin specifically cleaves a different amino acid bond of a SNARE protein. For example, the tetanus neurotoxin and the botulinum neurotoxin types B, D, F, and G degrade synaptobrevin (also known as “vesicle-associated membrane protein” or VAMP). Botulinum toxin type B cleaves VAMP at Gln76-Ph77. Botulinum toxin type D cleaves VAMP at Lys59-Leu-60. Botulinum toxin type F cleaves VAMP at Leu58-Lys59. And, botulinum toxin type G cleaves VAMP at a single Ala-Ala bond. VAMP is a synaptosomal membrane protein that is essential for vesicle release. Most of the VAMP present at the cytosolic surface of the synaptic vesicle is removed as a result of any one of the above cleaving events.

Similarly, botulinum neurotoxin types A and E block the release of ACh by cleaving a synaptosome-associated protein of molecular weight 25 kilodaltons, also known as SNAP-25. Botulinum toxin type A cleaves SNAP-25 at Gln197-Arg198, and botulinum toxin type E cleaves SNAP-25 at Arg180-Ile181. SNAP-25 is a plasma membrane protein that is located on the internal side of the plasma membrane of presynaptic nerve cells. SNAP-25 is integral to the vesicle release process. It is believed that the potency and duration of action of toxin type A derive, at least in part, from its action on SNAP-25. See Billante, C R., Muscle & Nerve, 26:395-403 (2002).

Botulinum neurotoxin type C also cleaves SNAP-25. In addition, type C also cleaves the protein syntaxin. Syntaxin is a presynaptic membrane protein that is associated with calcium channels and SNAP-25. Botulinum neurotoxin type C is a zinc-endopeptidase that cleaves syntaxin isoform 1A at the Lys253-Ala254 peptide body and syntaxin isoform 1B at the Lys252-Ala253 peptide bond, only when they are inserted into a lipid bilayer. Syntaxin isoforms 2 and 3 are also cleaved by Botulinum neurotoxin type C. However, syntaxin isoform 4 is resistant to botulinum neurotoxin type C cleaving. See Schiavo G., J. Biol. Chem., 5:270(18): 10566-70 (1995).

The cleavage of all of these proteins prevents fusion of the vesicles with the terminal nerve membrane. This, in turn, prevents the release of neurotransmitters (e.g., ACh) into the neuromuscular junction or synapse. It should be noted that while neurotoxins like botulinum toxin type A, prevent the release of ACh, they do not affect its synthesis or storage in the presynaptic neuron. Furthermore, they do not affect the conduction of electrical signals by such cells.

While the above neurotoxins denervate the presynaptic cells, evidence indicates that the presynaptic cell actually expands its endplate region in response to neurotoxins. For example, it has been shown that recovery from the effects of botulinum toxin type A is in part due to sprouting of new axons around toxin-blocked receptors to reestablish neuromuscular pathways. See Billante, C. R., et al., Muscle & Nerve, 26: 395-403 (2002). In particular, recovery from botulinum toxin type A has been found to have two distinct phases. In the first phase, recovery is due to neuron reinnervation by sprouting. In the second phase, neuron innervation is a result of unblocking of the original nerve terminals with retraction of nerve sprouting. Id.

Thus, the present invention also contemplates the inhibition of neurotransmission by administering one or more neurotoxins and one or more neuron growth inhibitors to a target region. The term “neuron growth inhibitor” as used herein refers to any substance that inhibits, interferes with, reduces, or decreases neuron and/or axonal growth (e.g., sprouting). Thus, a neuron growth inhibitor can be useful in increasing the efficacy of a neurotoxin by delay repair of a neurojunction, for example.

In preferred embodiments, a neuron growth inhibitor is any substance that interferes with the MAPK pathway or its activation of MEK/ERK. MAPK has been suggested to be involved in synaptic plasticity in post-mitotic cells of the central nervous system (CNS). For example, some studies suggest that MAPK is necessary for long-term facilitation of Aplysia sensory neuron-motor neuron synapses, associative conditioning in Hermissenda, and hippocampal long-term potentiation in rodents. See Adams, J. P., Neural Notes, Vol. 1, Issue 1 (1999. The MAPK cascade is regulated by a succession of kinases. A typical signal transduction pathway via MAPK is illustrated in FIG. 1. In FIG. 1, a growth factor (GF) (e.g., epidermal growth factor (EPG) or neuronal growth factors (NGF)) binds its growth factor receptor (GFR) on the cell surface. Growth factor receptors are generally tyrosine kinase (Trk) receptors.

There are three types of Trk receptors, each of which can be activated by one or more of the following four neurotrophis: NGR, brain-derived neurotrophic factor (BDNF), and neurotrophins 3 and 4 (NT3 and NT4). See Huang, E J., Annual Review of Biochemistry, Vol. 72, p. 609-642 (2003). Neurotrophin signaling through these receptors regulates, in part, cell survival, proliferation, and axon and dendrite growth and patterning. Id. Another type of receptor that function as signal transductors of neurotrophins is p75NTR. P75NTR is a member of the TNF receptor superfamily and is an effector of NF-KB.

In general, the binding of a growth factor to its Trk receptor causes that receptor to dimerize with an identical receptor. This dimerization initiates an autophosphorylation of tyrosine residues on the intracellular tail of the dimerized receptors. The phosphotyrosines that result from the autophosphorylation function as docking sites for signaling molecules such as Grb2 (an adaptor protein), SOS (a guanine nucleotide exchange factor) and Ras (a GTP binding protein). Other molecules that are activated by Trk receptors include Rap-1, and the Cdc-42-Rac-Rho family, PI3K, and phospholipase-C-gamma.

In particular, the Grb2-SOS complex activates the small G-protein, Ras, by stimulating the exchange of guanosine diphosphate (GDP) for guanosine triphosphate (GTP). Other activators of Ras include, but are not limited to Phospholipase C and calmodulin (e.g., in response to calcium influx). Rit and Rin and two homologous Ras-like proteins that are plasma membrane-localized. Rin binds calmodulin through a C-terminal binding motif. It has been suggested that Rit and Rin define a novel subfamily of Ras-related proteins, perhaps using a new mechanism of membrane association, and that Rin may be involved in calcium-mediated signaling within neurons. See Lee, C H J, et al., The Journal of Neuroscience, Vol. 16, No. 21, pp. 6784-6794 (1996).

Activation of Ras is associated with the promotion of cell proliferation (mediation of growth factors), cell differentiation (e.g., PC12 cells), and differentiation of cell functions (mediate calcium signaling). Ras is a notable member of the large family of GTPases, proteins that bind and hydrolyze GTP. The Ras superfamily, which includes approximately 50 different members, can be divided into subfamilies according to function and sequence. One subfamily is associated with cell growth and differentiation includes the following members: H-Ras, N-Ras, K-Ras, TC-21, Rap-1, Rap-2, R-Ras, Ral-A, Ral-B. Another Ras superfamily associated with cytoskeleton structuring includes the following members: Rho-A, Rho-B, Rho-G, Rho-E, CDC-42, Rac-1, and Rac-2. A third Ras superfamily associated with vesicle sorting includes the following members: Rab, Arf, and Ran.

Effectors of Ras include but are not limited to phosphatidylinositol-3′-kinase (PI3K), Raf, and Ral. Over-expression of P13K is associated with enlarged cell somata and axon width. Id. It is also a known activator of Atk, a serine/threonine kinase that is essential for growth dependent survival of neurons. See Markus A., Neuron, Vol. 35: 65-76 (2002). Over-expression of Atk is associated with an in increase in the number of axon branches as well as enlargement of the cell somata. Id.

Ras activates MEK and ERK by a central three-tiered core signaling module, which comprises of an apical MAPK kinase kinase (MAP3K), a MAPK kinase (MEK or MKK), and a downstream MAPK. MEK in turn phosphorylates and activates extracellular-signal-regulated kinase (ERK).

The most common MAP3K is Raf. The exchange of guanosine diphosphate (GDP) for guanosine triphosphate (GTP) by Ras elicits a conformational change, which enables it to bind and activate Raf. The Raf kinase family is a serine/threonine protein kinase which catalyzes hydroxyl groups on specific serine and threonine residues. Interaction of Ras with Raf is thought to be necessary but not sufficient to activate Raf. Mammals possess 3 Raf proteins, ranging from 70 to 100 kDa in size. These Raf isomers are known as: a-Raf, b-Raf, and c-Raf or Raf-1. While Raf-l is ubiquitously distributed throughout the body, a-Raf is found abundantly in urogenital tissue and b-Raf is found predominantly in neuronal tissue.

Generally, Ras recruits Raf (e.g., b-Raf or Raf-1) from the cytosol to the cell membrane, where Raf is activated. Raf activation is thought to involve a multi-step process that includes the dephosphorylation of inhibitory sites by protein phosphatase 2A (PP2A) and the phosphorylation of activating sites by PAK (p21^{rac/cdc-12}-activated kinase), Src-family and some unknown kinases.

B-Raf kinases are associated with extracellular signaling that suppress apoptosis and regulate cell differentiation. Additional activators of b-Raf include, PKA, PKB, PKC, KSR, Pak, and 14-3-3. While PKA inhibit Raf-1 catalytic activity in most cells, it potentiates nerve growth factor-stimulated PC12 cell differentiation, which is a b-Raf mediated process. This potentiation rather than inhibition of PC12 cell differentiation is thought to be the result of the N-terminal regulatory domain of PKA. It is believed that this domain interferes with the ability of PKA to modulate b-Raf catalytic activity and provides resistance of b-Raf-dependent processes to PKA inhibition.

Rheb (Ras homolog enriched in brain) is a new class of G-proteins and is a member of the Ras superfamily and an immediate member of the Rap/Ral subfamily. Rheb, like Ras and Rap1, binds b-Raf kinase, but in contrast to Ras and Rap 1, Rheb inhibits b-Raf kinase activity and prevents b-Raf-dependent activation of the transcription factor Elk-1. Rheb homologs can be define based on their overall sequence similarity, high conservation of their effector domain sequence, presence of a unique arginine in their G1 box, and presence of a conserved CAAX farnesylation motif.

MEK is a unique kinase in that it phosphorylates MAPK on both threonine and tyrosine residues. MEK is the only known activator of MAPK, and MAPK is the only known target of MEK. Activated ERK has many substrates in the cytosol (e.g. cytoskeletal proteins, such as MAP and Tau, nuclear transcription factors such as Elk, Myc, Fos, and Jun, signaling molecules such as cytosolic phospholipase A2, and other kinases such as RSK. Id. Inhibition of MEK/MAPK and p53 pathways has been associated with nerve growth inhibition. See Pumiglia, K. M., Proc. Natl. Acad. Sci. USA, Vol. 94, 448-452 (January 1997); see also Adams, J. P., Neural Notes, Vol. V, Issue 1, 14-16 (1999); see also Mazzoni, I. E., J. Neurosci. 19(22): 9716-27 (November 1999).

In neuronal cell lines such as PC12, NGF and EGF have been shown to use the same Raf/MEK/ERK pathway to cause PC12 proliferation and differentiation. However, another pathway exists to induce neuronal endplate growth. This pathway is activated by the pituitary adenylate cyclase-activating polypeptide (PACAP). See Vaudry, D., Science, Vol 296: 1648-49 (2002). PACAP has been found to cause robust neurite outgrowth by activating ERK. PACAP signaling is believed to be independent of Ras. PACAP is thought to activate adenylate cyclase (AC), which increases intracellular cAMP. cAMP, in turn, activates ERK through PKA. However, inhibition of PKA with H89 does not seem to block activation of ERK. Id. This suggests that cAMP may activate ERK through the Raf/MEK/ERK pathway, e.g., via Rap-1 or another effector.

Thus, the present invention contemplates the use of a neuron growth inhibitor in combination with a neurotoxin for the treatment and/or prevention of various conditions. In preferred embodiments, a neuron growth inhibitors is selected from the group consisting of a Trk receptor inhibitor, a Ras inhibitor, a Raf kinase inhibitor, a Rap-1 inhibitor, a PKA inhibitor, a p53 inhibitor, a MEK inhibitor, an ERK inhibitor, a NF-KB inhibitor, am inhibitor of a growth factor (e.g., NGF), or an inhibitor of an isozyme, derivative, splicing variant, activator or effector (target) of any of the above (e.g., Ras, Raf, Rap-1, etc.).

Examples of MEK inhibitors include but are not limited to SL327, PD98059 (CalBiochem Cat. No. 513000), U0126 (CalBiochem Cat. No. 662005), PD 184352 (see Delaney, A. M., Molec. Cell Biol., Vol. 22, No. 21, p. 7593-7602 (2002); 2-Cholor-3-(N-succinimidyl)-1,4-naphthoquinone (CalBiochem Cat. No. 444938), ARRY-142886 (AstraZeneca), tricyclic flavone, and 2-(2-amino-3-methoxyphenyl)-4-oxo-4H-[1]benzopyran.

Examples of Ras inhibitors include, but are not limited to, N17Ras and farnesyltransferase inhibitors (FTIs), such as FTI-277 and nontoxic farnesylcysteine analogue farnesylthiosalicylic acid (FTS), which dislodges all Ras isoforms from the membrane. See Kloog, Y., Mol. Med. Today, 6(10): 398-402 (2000); see also Aletsee, C., JARO, (02) 377-378 (2001).

Examples of P13K inhibitors include, but are not limited to, LY294002.

Examples of compounds that inhibit the Raf-Ras interaction include, but are not limited to, those short peptides disclosed in Zeng, J., Protein Engineering, Vol. 14, No. 1, 39-45, (2001) and MCP1 and its derivatives, 53 and 110 (see Kato-Stanlidewicz, J., Proc. Natl. Acad. Sci. USA., 99 (22): 14398-14403 (2002)).

Examples of b-Raf inhibitors include, but are not limited to, bis-aryl ureas, such as, e.g., BAY-43-9006, which inhibit b-Raf (see Wilhelm S., Current Pharmaceutical Design, Vol. 8, No. (2002)), Rheb (Ras homolog enriched in brain), which inhibits b-Raf, and RKIP (Raf kinase inhibitor protein).

An example of a PKA inhibitor is H-89.

Examples of PKC inhibitors include competitive inhibitors for the PKC ATP-binding site, including staurosporine and its bisindolylmaleimide derivitives, Ro-31-7549, Ro-31-8220, Ro-31-8425, Ro-32-0432 and Sangivamycin; drugs which interact with the PKC's regulatory domain by competing at the binding sites of diacylglycerol and phorbol esters, such as calphostin C, Safingol, D-erythro-Sphingosine; drugs which target the catalytic domain of PKC, such as chelerythrine chloride, and Melittin; drugs which inhibit PKC by covalently binding to PKC upon exposure to UV lights, such as dequalinium chloride; drugs which specifically inhibit Ca-dependent PKC such as Go6976, Go6983, Go7874 and other homologs, polymyxin B sulfate; drugs comprising competitive peptides derived from PKC sequence; and other PKC inhibitors such as cardiotoxins, ellagic acid, HBDDE, 1-O-Hexadecyl-2-O-methyl-rac-glycerol, Hypercin, K-252, NGIC-I, Phloretin, piceatannol, Tamoxifen citrate. Additional inhibitors shown to be effective include: 542 (+−)-1-(5-Isoquinolinesulfonyl)-2-methylpiperazine dihydrochloride; IC50=6.0 μM; 543 1-(5-Isoquinolinesulfonyl)piperazine; IC50=6.0 μM; 609 (+/−)-Palmitoylcarnitine chloride; 621 10-[3-(1-Piperazinyl)propyl]-2-trifluoromethylphenothiazine dimaleate; 632 (+/−)-Stearoylcamitine chloride. Alternative pharmacologically acceptable inhibitors effective in the disclosed methods are readily screened from the wide variety of PKC inhibitors known in the art (e.g Goekjian et al., Expert Opin. Investig. Drugs, 10, 2117-40 (2001); see also Battaini, Pharmacolog. Res., 44, 353-61 (2001). See U.S. Pat. No. 6,664,266, assigned to Children's Medical Center Corporation, incorporated herein by reference for all purposes.

An example of a Rap-1 inhibitor is SB203580.

In some embodiments, the invention herein utilizes a MEK inhibitor such as PD98059 to inhibit or delay neurojunction repair. In some embodiments, the invention herein utilizes a Raf kinase inhibitor, or more preferably, a b-Raf kinase inhibitor (e.g., Rheb or BAY-43-9006) to inhibit or delay neurojunction repair.

In addition, a neuron growth inhibitor of the present invention may also be an antisense, an antibody, a small or large organic or inorganic molecule, or any other compound that reduces or arrests the growth of nerve cells.

The term “antisense oligonucleotide” or “antisense,” as used herein, describes composition that include a nucleic acid sequence which specifically hybridizes under physiological conditions to a target p38 or RNA thereby inhibiting its transcription and/or translation. Antisense oligonucleotides include siRNA. (See Liang Y, et al., Clin Cancer Res. 2003:19(16 suppl):77. Abstract All l.) Antisense oligonucleotides can comprise of oligoribonucleotide, oligodeoxyribonucleotide, modified oligoribonucleotide, or modified oligodeoxyribonucleotide, and any derivatives, variants, fragments, and/or mimetics thereof. Antisense oligonucleotides can be naturally occurring or synthetic.

Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions.

Thus in preferred embodiments, an antisense oligonucleotide can specifically hybridize with p38 or RNA of b-Raf, Ras, Rap-1, MEK, PKA, P13-K, Akt, p53, ERK, a growth factor, (e.g., NGF), any elements that are upstream or downstream in the MAPK/MEK/ERK pathway or p53 pathway, and/or any derivative, variant, mimetic, or fragment of any of the above.

The term “antibody” or “antibodies,” as used herein, refers to any immunoglobulin that binds specifically to an antigenic determinant. Examples of antibodies include, but are not limited to, monoclonal antibodies, polyclonal antibodies, humanized antibodies, chimeric antibodies, single chain antibodies, Fab fragments, F(ab′)₂ fragments, fragments produced by FAb expression library, anti-idiotypic (anti-Id) antibodies, epitope-binding fragments of any of the above. Antibodies can be any immunoglobulin (e.g., IgG, IgM, IgA, IgE, IgD, etc.) obtained from any source (e.g., humans, rodents, non-human primates, lagomorphs, caprines, bovines, equines, ovines, etc.). In some embodiments, an antibody is directed against a species (e.g., anti-mouse, anti-human, etc.).

In preferred embodiments, a neuron growth inhibitor can be an antibody, more preferably a monoclonal antibody, or more preferably a chimeric or humanized antibody. Such antibody can preferably specifically bind to any one of the proteins that enhances neuronal growth or collateral axonal sprouts.

For example, the present invention contemplates a neuronal growth inhibitor that is an antibody that can specifically bind to Raf, Ras, Raf, MEK, P13-K, Akt, p53, ERK, a growth factor, (e.g., NGF), any elements that are upstream or downstream in the MAPK/MEK/ERK pathway or p53 pathway, and/or any derivative, variant, mimetic, or fragment of any of the above. In preferred embodiments, a neuronal growth inhibitor is a monoclonal antibody that can specifically bind to MEK or ERK or Raf or b-Raf.

The present invention contemplates the use of at least one neurotoxin and/or at least one neuron growth inhibitor for the treatment and prevention of a disease. Such a disease can include by way of example, any neurological, neuromuscular, urological, dermatological, and optical condition. Such conditions may further be characterized by involuntary muscle spasms, chronic pain, and/or aging skin.

In some embodiments, the present invention contemplates the use of at least one neurotoxin and/or at least one neuron growth inhibitor for the treatment and prevention of any condition for which a neurotoxin is used as a therapeutic agent. For example, today, botulinum toxin type A is approved for use for brow wrinkle removal, blepharospasm, strabismus, and Duane's syndrome. Blepharospasm is a condition associated with uncontrollable twitching of an eyelid that can be benign and/or related to stress, sleep deprivation, or the use of stimulants. Strabismus is an eye disorder wherein the optic axes cannot be directed to the same object. Duane's syndrome is a hereditary congenital syndrome in which the affected eye shows limitation or absence of abduction, restriction of adduction, retraction of the globe on adduction, narrowing of the palpebral fissure on adduction and widening on adduction, or deficient convergence.

Thus, the present invention contemplates the use of at least one neurotoxin and/or at least one neuron growth inhibitor for the treatment or prevention of dermatological and optical conditions such as brow wrinkle removal, blepharospasm, strabismus, and Duane's syndrome. Administration of the neurotoxin and/or the neuron growth inhibitor are preferably made locally (e.g., topically, subdermally, intramuscularly, or subcutaneously). In a combination treatment, the neurotoxin may be administered prior to, simultaneous with, or after the administration of the neuron growth inhibitor. In preferred embodiments, the neurotoxin is administered prior to the administration of the neuron growth inhibitor.

Neurotoxins may also be used for the treatment or prevention of localized dystonia. Examples of localized dystonia include, but are not limited to, cervical dystonia, embouchure dystonia, oromandibular dystonia, spasmodic dystonia, and writer's cramp. Cervical dystonia, also known as spasmodic torticollis, is a localized dystonia that is characterized by neck muscles contracting involuntarily. This may result in abnormal movements and posture of the head and neck. Embouchure dystonia is a term used to describe a type of dystonia that affects brass and woodwind players. Embouchure dystonia causes excessive twitching of the lips and may also cause forceful contractions of the jaw and tongue. Thus, patients suffering from oromandibular dystonia may experience difficulty in opening and closing their mouths as well as chewing and speaking. Spasmodic dystonia involves involuntary “spasms” of the vocal cords which may cause interruptions in speech and changes in voice quality. Furthermore, writer's cramp is a form of a localized dystonia, which is task specific and usually affects the hand and/or the arm.

Thus, in some embodiments, the present invention contemplates administration of at least one neurotoxin and/or at least one neuron growth inhibitor for the treatment of a localized dystonia. For example, a dystonia such as cervical dystonia, embouchure dystonia, oromandibular dystonia, spasmodic dystonia, and writer's cramp dystonia relay be treated by administering locally to a target region at least one neurotoxin and at least one neuron growth inhibitor. Preferably, a neurotoxin is administered prior to the administration of the neuron growth inhibitor.

Additional indications that may be treatable or preventable by the compositions and methods herein are neurological disorders. Such neurological disorders include, but are not limited to, migraine headache, chronic pain (e.g., chronic low back pain), chronic muscle pain (e.g., fibromyalgia), stroke, traumatic brain injury, localized pain (e.g., vulvodynia), cerebral palsy, meige syndrome, hyperhydrosis, tremor, achalasia, secondary and inherent dystonias, Parkinson's disease, spinal cord injury, multiple sclerosis, and spasm reflex.

The compositions and methods herein may be especially useful in the treatment and prevention of urological conditions. Examples of urological conditions include, but are not limited to, pelvic pain (e.g., interstitial cystitis, endometriosis, prostatodynia, urethral instability syndromes), pelvic myofiscial elements (e.g., levator sphincter, dysmenorrhea, anal fistula, hemorrhoid), urinary incontinence (e.g., unstable bladder, unstable sphincter), prostate disorders (e.g., prostatic hyperplasia, benign prostatic hyperplasia, prostatic enlargement, BPH prostatitis, prostate cancer), recurrent infection (secondary to sphincter spasticity), and urinary retention (secondary to spastic sphincter, hypertrophied bladder neck) and bladder dysfunction.

In some embodiments, the compositions and methods herein may be used to treat and prevent skin condition and/or enhance wound healing. Exemplary skin conditions include eczema, psoriasis, dermatitis, melonoma, pityriasis, such as pitiyriasis rosea, pityriasis rosacea and pityriasis rubra, and other cutaneous cell-proliferative disorders. Skin wounds include, for example, facial or bodily lacerations, whether elective (e.g., surgically introduced incisions) or non-elective (e.g., lacerations caused by car accident).

In some embodiments, the compositions and methods herein may be used to treat or prevent injury to the muscle. Examples of muscle injuries include, but are not limited to, contusions (bruises), lacerations, ischemia, strains, and complete ruptures.

In some embodiments, the compositions and methods herein may be used to treat thyroid disorder such as hyperthyroidism, hypothyroidism, Graves' disease, goiter, thyroiditis, cancer, and all other conditions that may result in hypothyroidism or hyperthyroidism.

In some embodiments, the compositions and methods herein can be used to suppress or reduce snoring noises.

Those of ordinary skill in the art will know, or can readily ascertain, how to obtain the neurotoxins of the invention, including the botulinum and tetanus toxins, in a pharmaceutically safe form. Such form is preferably nonteratogenic and does not induce a detectable immune response to the toxin antigen. For most of the neurotoxins of the invention, pharmaceutical safety will be dose-dependent such that relatively low dosages of toxin will be “safe” as compared to dosages which are known to be sufficient to produce disease.

Preferably, the neurotoxins and/or neuron growth inhibitors of the invention will be administered as a composition in a pharmaceutically acceptable carrier. To that end, presynaptic neurotoxin compositions and/or neuron growth inhibitors are prepared for administration by mixing a toxin the desired degree of purity with physiologically acceptable sterile carriers. Such carriers will be nontoxic to recipients at the dosages and concentrations employed. Ordinarily, the preparation of such compositions entails combining the neurotoxin with buffers, antioxidants such as ascorbic acid, low molecular weight (less than about 10 residues) polypeptides, proteins, amino acids, carbohydrates including glucose or dextrins, chelating agents such as EDTA, glutathione and other stabilizers and excipients. Such compositions may also be lyophilized and will be pharmaceutically acceptable; i.e., suitably prepared and approved for use in the desired application.

A pharmaceutical composition of the present invention may be formulated to be suitable for application in a variety of maimers, for example, in a cream for topical application to the skin (e.g., for alopecia), in a wash, in a douche, in a powder for chaffing (e.g., for dermatitis), in a liquid, in a dry formulation (e.g., as a bath salt or bath powder), and the like. Other formulations will be readily apparent to one skilled in the art. In preferred embodiments, the compositions herein are preferably formulated for local administration. Preferably, the compositions are formulated for topical, subcutaneous, intramuscular, or transdermal administration.

For transdermal and topical administration, the neurotoxins and/or neuron growth inhibitors will preferably be formulated to enhance penetration to and across the stratum corneum of the skin. Those of ordinary skill in the art will be familiar with, or can readily ascertain the identity of, excipients and additives, which will facilitate drug delivery (particularly of peptides) across skin. For review in this respect, reference may be made to “Novel Drug Delivery Systems”, Chien, ed. (Marcel Dekker, 1992), the disclosure of which is incorporated herein by this reference to illustrate the state of knowledge in the art concerning drug delivery to and across the stratum corneum of skin.

When formulated as an ointment, the active ingredient (e.g., the neurotoxins and/or neuron growth inhibitors) can be employed, for example, with either paraffinic or a water miscible ointment base. Alternatively, the active ingredients can be formulated in a cream with an oil-in-water cream base. If desired, the aqueous phase of the cream base can include, for example at least 30% w/w of a polyhydric alcohol such as propylene glycol, butane-1,3-diol, mannitol, sorbitol, glycerol, polyethylene glycol and mixtures thereof.

A topical formulation can desirably include a compound that enhances absorption or penetration of the active ingredient through the skin or other affected areas. Examples of such dermal penetration enhancers include dimethylsulfoxide and related analogs. Topical formulation may further include, for example, antioxidants (e.g., vitamin E); buffering agents; lubricants (e.g., synthetic or natural beeswax); sunscreens (e.g., para-aminobenzoic acid); and other cosmetic agents (e.g., coloring agents, fragrances, oils, essential oils, moisturizers or drying agents). Thickening agents (e.g., polyvinylpyrrolidone, polyethylene glycol or carboxymethyicellulose) may also be added to the compositions.

The carriers utilized in the pharmaceutical compositions of the present invention may be solid-based dry materials for use in powdered formulations or may be liquid or gel-based materials for use in liquid or gel formulations. The specific formulations depend, in part, upon the routes or modes of administration.

Typical carriers for dry formulations (e.g., bath salts) include, but are not limited to, trehalose, malto-dextrin, rice flour, micro-crystalline cellulose (MCC), magnesium sterate, inositol, fructo-oligosaccharides FOS, gluco-oligosaccharides (GOS), dextrose, sucrose, talc, and the like carriers. Where the composition is dry and includes evaporated oils that produce a tendency for the composition to cake (i.e., adherence of the component spores, salts, powders and oils), it is preferable to include dry fillers which both distribute the components and prevent caking. Exemplary anti-caking agents include MCC, talc, diatomaceous earth, amorphous silica and the like, typically added in an concentration of from approximately 1% to 95% by-weight.

Suitable liquid or gel-based carriers are well-known in the art (e.g., water, physiological salt solutions, urea, methanol, ethanol, propanol, butanol, ethylene glycol and propylene glycol, and the like). Preferably, water-based carriers are approximately neutral pH.

Additional suitable carriers include aqueous and oleaginous carries such as, for example, white petrolatum, isopropyl myristate, lanolin or lanolin alcohols, mineral oil, fragrant or essential oil, nasturtium extract oil, sorbitan mono-oleate, propylene glycol, cetylstearyl alcohol (together or in various combinations), hydroxypropyl cellulose (MW=100,000 to 1,000,000), detergents (e.g., polyoxyl stearate or sodium lauryl sulfate) and mixed with water to form a lotion, gel, cream or semi-solid composition. Other suitable carriers comprise water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether and water. For example, emulsions containing water, glycerol stearate, glycerin, mineral oil, synthetic spermaceti, cetyl alcohol, butylparaben, propylparaben and methylparaben are commercially available. Preservatives may also be included in the carrier including methylparaben, propylparaben, benzyl alcohol and ethylene diamine tetraacetate salts. Well-known flavorings and/or colorants may also be included in the carrier. The composition may also include a plasticizer such as glycerol or polyethylene glycol (MW 400 to 20,000). The composition of the carrier can be varied so long as it does not interfere significantly with the pharmacological activity of the active ingredient (botulinum toxin type A).

When administering a neurotoxin (especially botulinum toxin type A) and/or a neuron growth inhibitor, small dosages should be applied. Generally, the dose of the neurotoxin and/or neuron growth inhibitor to be administered will vary depending on the age of the host being treated, sex and weight of the host, condition being treated, severity of such condition, location of the condition, and potency of the neurotoxin.

Toxin potency is expressed as a multiple of the LD₅₀ value for a reference mammal, usually a mouse. Where a mouse is the reference mammal, one “unit” of toxin is the amount of toxin that kills 50% of a group of mice that were disease-free prior to inoculation with the toxin. For example, commercially available botulinum toxin A typically has a potency such that one nanogram contains about 40 mouse units. It should also be noted that each neurotoxin, neurotoxin type, and/or neuron growth inhibitor may have its own LD₅₀ and that the LD₅o may vary depending on the animal species. The potency in humans of the botulinum toxin type A product supplied by Allergan, Inc. is believed to be about LD₅₀=2,730 units. Furthermore, it has been shown that botulinum toxin type A is 500 times more potent, as measured by the rate of paralysis produced in the rat, than is botulinum toxin type B.

Assuming a potency which is substantially equivalent to LD₅₀=2,730 units, the neurotoxin can be administered in a dose of up to about 2000 units, although individual dosages will be smaller. For example, a single application in a treatment cycle can include 0.25-50 units of a neurotoxin, more preferably 0.5-25 units of a neurotoxin, more preferably 1 to 10 units of a neurotoxin, more preferably 1.25-5 units of a neurotoxin, or more preferably 1.25-2.5 units of a neurotoxin. The present invention also contemplates administering smaller doses of a neurotoxin (especially in combination treatments). Such doses may be less than 5 units of a neurotoxin per application, less than 2 units of a neurotoxin per application, less than 1 units of a neurotoxin per application, or less than 0.5 units of a neurotoxin per application.

The above dosages for neurotoxins may be administered once or at recurring intervals or on an as need basis. For example, the above dosages may be administered once a day, more preferably about once a week, more preferably about once a month, more preferably about every 3 months, more preferably about every 6 months, or more preferably about every 9 months. Greater time intervals are also contemplated by the present invention. The dosage may also be adjusted upward or downward depending additional agents administered (e.g., a neuron growth inhibitor), the condition and severity of condition being treated, and the sex, age and specie of mammal being treated. Preferably, the lowest therapeutically effective dosage will be administered. In the initial treatment, a low dosage may be administered at a target site to determine the patient's sensitivity to, and tolerance of, the neurotoxin. Additional injections of the same or different dosages will be administered as necessary.

Thus, an effective amount of a neurotoxin is a dosage sufficient to delay, decrease, interfere, or inhibit neuronal transmission for at least one day, more preferably for at least one week, more preferably for at least one month, more preferably for at least 3 months, more preferably for at least 6 months, more preferably for at least 9 months, or more preferably for at least 1 year.

In any of the embodiments herein, a neuron growth inhibitor may be administered in addition to the neurotoxin. A combination treatment of a neuron growth inhibitor and a neurotoxin involves administering both an effective amount of at least one neurotoxin and an effective amount of at least one neuron growth inhibitor. When administering a combination treatment, the effective amount of either or both the neurotoxin and/or the neuron growth inhibitor may be less than in a single drug therapy due to the synergistic effect of both agents. A combination treatment of a neurotoxin and a neuron growth inhibitor can include administration of a neurotoxin prior to, contemporaneous with, or post administration of a neuron growth inhibitor. For example, a neuron growth inhibitor may be administered simultaneous to, immediately subsequent to, approximately 5 minutes subsequent to, about an hour subsequent to, about 2 hours subsequent to, about 6 hours subsequent to, about a day subsequent to, about 2 days subsequent to, about a week subsequent to, about 2 weeks subsequent to, about a month subsequent to, about 3 months subsequent to, or about 6 months, subsequent to a neurotoxin threatening. In preferred embodiments, the neuron growth inhibitor is administered simultaneous to or immediately following the administration of a neurotoxin.

In some embodiments, an effective amount of a neuron growth inhibitor is the dosage sufficient to delay, decrease, interfere, and/or inhibit neuronal and/or axonal growth (e.g., sprouting) for at least one day, more preferably for at least one week, more preferably for at least one month, more preferably for at least 3 months, more preferably for at least 6 months, more preferably for at least 9 months, or more preferably for at least 1 year.

In some embodiments, an effective amount of a neuron growth inhibitor is the dosage sufficient to delay, decrease, interfere, and/or inhibit neurotransmission for at least one day, more preferably for at least one week, more preferably for at least one month, more preferably for at least 3 months, more preferably for at least 6 months, more preferably for at least 9 months, or more preferably for at least 1 year.

Dosing of either or both the neurotoxin and/or neuron growth inhibitor can be single dosage or cumulative (serial dosing), and can be readily determined by one skilled in the art. For serial dosing (i.e., one dose per day, more preferably one dose per week, more preferably one dose per month, more preferably one time per every six months, more preferably one time per every eight months, or more preferably one time per every one year), a dosage schedule can be readily determined by one skilled in the art based on, e.g., patient size, condition to be treated, severity of the condition, neurotoxin selected, and other variables.

One suggested course of treatment and/or prevention involves the use of a neurotoxin (e.g., botulinum toxin type A) and a neuron growth inhibitor (e.g., a MEK inhibitor). The neurotoxin is administered at about 40 units every three days up to the LD₅₀ for the neurotoxin. More preferably, the neurotoxin is administered at about 20 units every three days up to the LD₅₀ for the neurotoxin. More preferably, the neurotoxin is administered at about 10 units every three days up to the LD₅₀ for the neurotoxin.

In some embodiments, a neuron growth inhibitor may be administered in addition to (or in substitution to) the neurotoxin. A neuron growth inhibitor may be administered at a dosage rate of about 0.01 milligrams/kg per day to 2000 milligrams/kg per day, more preferably at a dosage rate of about 0.1 milligrams/kg per day to 1000 milligrams/kg per day, more preferably at a dosage rate of about 1 milligrams/kg per day to 750 milligrams/kg per day, more preferably at a dosage rate of about 5 milligrams/kg per day to 500 milligrams/kg per day, more preferably at a dosage rate of about 10 milligrams/kg per day to 250 milligrams/kg per day, more preferably at a dosage rate of about 25 milligrams/kg per day to 100 milligrams/kg per day, or more preferably at a dosage rate of about 30 milligrams/kg per day to 75 milligrams/kg per day. The dosage rate can change depending on the length of time between each application (e.g., 1 milligrams/kg per day is equivalent to about 7 milligrams/kg per week.) and the type of neurotoxin administered in conjunction with the neuron growth inhibitor.

The neuron growth inhibitor may be administered prior to, simultaneous with, or subsequent to the administration the neurotoxin. In preferred embodiments, the neuron growth inhibitor is administered subsequent to the administration the neurotoxin. For example, a neuron growth inhibitor may be administered ½ hour subsequent to the administration of a neurotoxin, more preferably 1 hour subsequent to the administration of a neurotoxin, more preferably 6 hours subsequent to the administration of a neurotoxin, more preferably 12 hours subsequent to the administration of a neurotoxin, more preferably 1 day subsequent to the administration of a neurotoxin, or more preferably 1 week subsequent to the administration of a neurotoxin.

The present invention contemplates the administration of a neurotoxin and a neuron growth inhibitor to treat and/or prevent various conditions. Predisposition to a condition may be determined prior to administration of the compositions herein according to conventional clinical standards, such as a prior or contemporaneous diagnosis or family history of the disease. Thus, a person diagnosed with a predisposition to a condition (especially one that is known to be treatable by a neurotoxin) may be administered a neurotoxin and a neuron growth inhibitor to prevent such condition.

For many indications, (especially those having a localized effect e.g., localized dystonia, psoriasis, wrinkles, etc.) subcutaneous, subdermal or intramuscular injections at the target site will be the most efficacious route of administration. Preferably, the injection will be provided to the subcutaneous or subdermal region beneath or into a target region (e.g., muscle effected by dystonia, or wrinkles) by inserting the needle below or into the target area. However, where a target region is too large or otherwise not susceptible to this approach, the compositions herein may be administered by transdermal or topical routes one or more target sites. However, it is expected that these latter routes will be less efficacious than subcutaneous, subdermal or intramuscular injections and may, therefore, be best used for subacute manifestations.

The injections will be repeated as necessary. As a general guideline, it has been observed that, after administration of a neurotoxin (e.g., botulinum toxin type A) into or near a target region in adult human skin according to the method of the invention, the treated region has remained paralyzed (e.g., neurotransmission has been inactivated) for periods of at least 2 months. Botulinum toxin type A in particular is expected to be most effective when administered according to the methods herein soon after the appearance of any indication of a condition. Depending on the course of therapy applied (i.e., with respect to dosage, frequency of treatment and sensitivity of individual patients to treatment), the method of the invention can be expected to be effective in mitigating the condition (e.g., reducing wrinkles or other alleviating pain), inducing remission of the condition, and in controlling symptoms associated with the condition (e.g., scaling of lesions and/or pain).

The neurotoxins and neuron growth inhibitors of the present invention are preferably administered locally. Local administration can be made, for example, by topical, subcutaneous, transdermal, subdermal or intramuscular administration.

In some embodiments, the methods of the present invention include administering to a mammal a combination treatment of a neurotoxin and one or more other agents that may interfere with neurotransmission, neuromuscular transmission, neuronal growth, and/or axonal growth (e.g., sprouting). A combination treatment may result in synergy between two or more compounds such that lower doses of individual compounds are required. A combination treatment may involve the simultaneous or sequential administration of two or more compounds. Therefore, according to the present invention, a neurotoxin may be administered with one or more neuron growth inhibitor simultaneously, or the neurotoxin may be administered prior to the administration of the neuron growth inhibitor, or the neurotoxin may be administered after the administration of a neuron growth inhibitor. In preferred embodiments, the neurotoxin is administered prior to the administration of the neuron growth inhibitor.

Administration of either or both the neurotoxin and the neuron growth inhibitor may be systemic or local. In preferred embodiments, either or both the neurotoxin and the neuron growth inhibitor are administered locally. Examples of localized administrations include, but are not limited to, topical, subcutaneous, subdermal, intramuscular, intracranial, vaginal, optical, anal, pulmonary, and transdermal administrations. In preferred embodiments, the administration of the compounds herein is made by topical, subcutaneous, subdermal, or transdermal administration. More preferably, administration of compounds herein is made by intramadcular or transdermal microinjections. However, needleless injections are also contemplated.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-152. (canceled)

153. Use of a p38 inhibitor for manufacturing a medicament for treating or preventing a condition characterized by the activation of the innate immune system wherein the condition is a skin or hair condition, and is selected from among: hair loss, vitiligo, and dermal scars.

154. A use of claim 153 where the p38 inhibitor is selected from among: pyridinylimidazoles, substituted pyrazoles, substituted pyridyls, quinazoline derivatives, aryl ureas, heteroaryl analogues, substituted imidazole compounds, and substituted triazole compounds.

155. Use of a p38 inhibitor for manufacturing a medicament for treating a skin condition selected from among vitiligo and dermal scarring, where the p38 inhibitor is administered topically.

156. A use of claim 155 wherein the dermal scarring is incident to acne.

157. Use of a composition containing a neurotoxin and a neuron growth inhibitor for manufacturing a medicament for treating a condition by inhibition of neurotransmission of a neurotransmitter.

158. A use of claim 157 wherein the neuron growth inhibitor inhibits neuron collateral sprouting.

159. A use of claims 157 or 158 where the condition is a selected from among localized dystonia, a thyroid condition, a neurological disorder, a muscle injury, a urological condition, an optical condition, a dermatological condition, a condition characterized by snoring, and a wound.

160. A use of claims 157, 158 or 159 where
a. the neurotoxin is selected from among botulinum toxin, tetanus toxin, curare, bungarotoxin, saxitoxin, and tetrodotoxin; and,
b. the neuron growth inhibitor is selected from among a Trk receptor inhibitor, a Ras inhibitor, a Raf kinase inhibitor, a Rap-1 inhibitor, an MEK inhibitor, an NF-kB inhibitor, an ERK inhibitor, a PKA inhibitor, a PKC inhibitor, a p53 inhibitor, and a growth factor inhibitor; and,
C. an inhibitor of an isozyme, derivative, splicing variant, activator or effector of any of the compositions set forth in subsection (b).
